SEQUENCE LISTING (11 sequences)

SEQ ID NO: 1 - DNA, 466 bp, Artificial Sequence
  equine IL-31
atgggctggtcctgcatcattctgtttctggtggccacagccaccggcgtgcactctggacctatctatcagctgcagcccaaagagatccaggccatcatcgtggaactgcagaacctgagcaagaagctgctggacgactacctgaacaaagaaaagggcgtgcagaagttcgacagcgacctgcctagctgcttcaccagcgattctcaggcccctggcaacatcaacagcagcgccatcctgccttacttcaaggccatctctcccagcctgaacaacgacaagagcctgtacatcatcgagcagctggacaagctgaacttccagaacgcccctgaaaccgaggtgtccatgcctaccgacaacttcgagcggaagcggttcatcctgaccatcctgcggtggttcagcaactgcctggaacacagagcccagcaccaccaccatcaccattgataagctt

SEQ ID NO: 2 - protein, 33 aa, Artificial Sequence
  equine IL-31 mimotope
NSSAILPYFKAISPSLNNDKSLYIIEQLDKLNF

SEQ ID NO: 3 - protein, 18 aa, Artificial Sequence
  feline IL-31 mimotope
AKVSMPADNFERKNFILT

SEQ ID NO: 4 - protein, 18 aa, Artificial Sequence
  equine IL-31 mimotope
TEVSMPTDNFERKRFILT

SEQ ID NO: 5 - protein, 18 aa, Artificial Sequence
  canine IL-31 mimotope
TEISVPADTFECKSFILT

SEQ ID NO: 6 - protein, 24 aa, Artificial Sequence
  equine IL-31 mimotope
GPIYQLQPKEIQAIIVELQNLSKK

SEQ ID NO: 7 - protein, 10 aa, Artificial Sequence
  equine IL-31 mimotope
KEKGVQKFDS

SEQ ID NO: 8 - protein, 165 aa, Artificial Sequence
  Feline_IL31_wildtype
MLSHAGPARFALFLLCCMETLLPSHMAPAHRLQPSDIRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQHHHHHH

SEQ ID NO: 9 - protein, 159 aa, Canis familiaris
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 10 - protein, 152 aa, Artificial Sequence
  Equine_IL31 polypeptide
MGWSCIILFLVATATGVHSGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHRAQHHHHHH

SEQ ID NO: 11 - protein, 155 aa, Artificial Sequence
  Equine_IL31-alternative polypeptide version
MVSHIGTTAFALFLLCCLGTLMFSHTGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHHHHHH

(19) United States
(12) Patent Application Publication
(10) Pub. No.: US 2022/0273793 A1
(43) Pub. Date: Sep. 1, 2022

(54) HORSE IL-31 INDUCED PRURITUS MODEL

(71) Applicant: Zoetis Services LLC, Parsippany, NJ (US)

(72) Inventors: Elizabeth A. VanBeelen, Mattawan, MI (US); Andrea J. Gonzales, Portage, MI (US); Timothy J. Fleck, Scotts, MI (US); Katelyn S. Vroman, Mattawan, MI (US); Gary F. Bammert, Portage, MI (US)

(21) Appl. No.: 17/674,991

(22) Filed: Feb. 18, 2022

Related U.S. Application Data
(60) Provisional application No. 63/151,962, filed on Feb. 22, 2021.

(51) Int. Cl. A61K 39/395; C07K 16/24; A61K 47/64; A61P 17/04

(52) U.S. Cl. CPC A61K 39/3955; C07K 16/244; A61K 47/646; A61P 17/04; A61K 2039/505

(57) ABSTRACT

The present invention provides an IL-31 horse pruritus model which confirms that IL-31 induces itch in horses and can be used to assess whether certain test compounds can block or reduce the itch in treated horses. This experimental model includes administering equine IL-31 to horses to produce a pruritic response; quantitatively measuring pruritic responses in the horses which were administered equine IL-31; administering a candidate horse IL-31 inhibitor; and assessing the effectiveness of the candidate horse IL-31 inhibitor in reducing pruritic behavior in the treated horses by challenging the horses with equine IL-31 following the administration of the candidate horse IL-31 inhibitor.

### CROSS-REFERENCE TO RELATED APPLICATIONS

This application claims the benefit of U.S. Provisional Application No. 63/151,962, filed Feb. 22, 2021, the entire contents of which are incorporated herein by reference in their entirety.

### FIELD OF THE INVENTION

The present invention relates to an IL-31 equine pruritus model for use in assessing the effectiveness of IL-31 inhibitor candidates in reducing pruritic behavior in the treated horses.

### BACKGROUND OF THE INVENTION

Pruritic skin conditions are common in equine medicine. Most pruritic conditions are due to allergic reactions to ectoparasites such as midges, flies or mites or environmental allergens such as pollens, barn dust, or molds. There are other causes of pruritus such as food allergies, atopic dermatitis or urticaria, as well as skin infections from staphylococci or fungi. (White S D Equine Veterinary Education 2015; 27(3) 156-166.

Insect-bite hypersensitivity (IBH) is the most common form of pruritic allergic dermatitis in horses worldwide. This condition is caused by an allergic reaction toward the saliva of biting midges (Culicoides), but other insects can also be involved. The animals become very pruritic, and papules, crusts and alopecia are found in affected areas.

Atopic dermatitis can also be found in horses. Atopic dermatitis has been defined by the American College of Veterinary Dermatology task force as “a genetically-predisposed inflammatory and pruritic allergic skin disease with characteristic clinical features” (Olivry, et al. Veterinary Immunology and Immunopathology 2001; 81: 143-146). Atopic dermatitis in horses is recognized as a potential cause of pruritus. The role of environmental allergens in equine atopic dermatitis is becoming better appreciated. The disease may be seasonal or non-seasonal, depending on the allergen(s) involved. Age, breed, and sex predilections have not been extensively reported. In preliminary work at the School of Veterinary Medicine, University of California, Davis (SVM-UCD), the median age at onset was 6.5 years, Thoroughbreds were the most common breed, accounting for 25% of the horses, and males (usually geldings) were almost twice as prevalent as mares; however, these data are from only 24 horses, and have not yet been compared with the hospital population at large. Pruritus, often directed against the face, distal legs, or trunk, is the most common clinical sign of equine atopic dermatitis. Alopecia, erythema, urticaria, and papules may all be present. Urticarial lesions may be quite severe, yet nonpruritic. There may be a familial predisposition for urticarial atopic dermatitis in the horse. Horses may have a secondary pyoderma, typified by excess scaling, small epidermal collarettes, or encrusted papules (“miliary dermatitis”). Diagnosis of atopic dermatitis is based on clinical signs and the exclusion of other diagnoses, especially insect (Culicoides) hypersensitivity (White, Clin Tech Equine Pract 2005; 4: 311-313; Fadok, Vet Clin Equine 2013; 29 541-550).

Currently, management of atopic dermatitis in horses is done both symptomatically, by suppressing the inflammation and the pruritus triggered by the allergic response, and by addressing the specific cause (i.e., by identifying the responsible allergens and by formulating an allergen-specific vaccine). The symptomatic approach is typically needed in the short term to make the patient comfortable and minimize self-trauma. This approach relies on the use of a combination of topical and systemic therapies including antihistamines, essential fatty acids, pentoxifylline, and glucocorticoids. The primary approach to environmental allergy control involves the identification of allergens that trigger the hypersensitivity reaction. It is commonly accepted by dermatologists that allergen-specific immunotherapy can be of help to atopic horses. However, as a general rule, most horses show improvement only after the first 6 months of immunotherapy (Marsella, Vet Clin Equine 2013; 29: 551-557). Also, long term use of immunosuppressive drugs in horses can result in undesirable adverse effects.

Equine allergic dermatitis is thought to involve a T helper cell type 2 (Th2) inflammatory response where T-cell cytokines such as interleukin (IL)-4, IL-13 and IL-31 are released from T helper type 2 (Th2) lymphocytes after allergen exposure and may contribute to the manifestations of clinical signs such as skin inflammation, pruritus, and hair loss (Heimann et al. Vet Immunol Immunopathol 2011; 140 (1-2): 63-74; McKelvie et al. Equine Vet J 1999; 31: 466-472) seen in these horses. IL-31 has been shown to induce pruritus in in mice, dogs, humans, and monkeys (Dillon et al. Nat Immunol 2004; 5:752-60; U.S. Pat. No. 8,790,651 to Bammert et al.; U.S. Pat. No. 10,526,405 to Mann et al.; Gonzales et al. Vet Dermatol 2013; 24(1): 48-53; Gonzales et al. Vet Dermatol. 2016; 27: 34-e10; Bieber N Engl J Med 2008; 358: 1483-1494; Lewis et al. JEADV 2017; 31: 142-150). Additionally, IL-31 levels are elevated in the serum of patients with pruritic allergic or atopic skin disease in humans (Lu et al., Journal of Central South University 2018; 43(2):124-130), dogs (Gonzales et al., 2013 supra) and cats (Dunham et al., Vet Dermatol 2018; 29: 284), suggesting IL-31 may be a key itch mediator in allergic skin disease across animal species. IL-31 binds a co-receptor composed of IL-31 receptor A (IL-31RA) and the oncostatin M receptor (OSMR) (Dillon et al. 2004 supra and Bilsborough et al. J Allergy Clin Immunol. 2006 117(2):418-25). Receptor activation results in phosphorylation of STAT through JAK receptor(s). Expression of the co-receptor has been shown in macrophages, keratinocytes and in dorsal root ganglia.

The prediction that targeting the IL-31 pathway may provide relief from itch has been confirmed in dogs. Cytopoint®, a canine anti-IL-31 monoclonal antibody (mAb) produced by Zoetis Inc., Parsippany, N.J., has been shown to reduce pruritus and skin lesions in dogs with atopic dermatitis (Gonzales et al. 2013 supra, Michels et al. Vet Dermatol. 2016; December; 27(6): 478-e129).

Researchers from the University of Florida have shown that IL-31 mRNA was upregulated in equine leukocytes isolated from allergic horses after re-exposure to their offending allergens, suggesting IL-31 may be secreted from immune cells when allergic horses are re-exposed to allergens (Craig et al. Abstracts of the North American Veterinary Dermatology Forum, April 10-13th 2019, Austin, Tex., USA. Vet Dermatol. 2019; 30(4):295).

A recent publication from the University of Zurich demonstrated a similar finding where peripheral blood mononuclear cells (PBMCs) from Icelandic horses expressed IL-31 mRNA in response to Culicoides nubecullosus allergen. They also showed IL-31 mRNA levels were elevated in the skin of horses with IBH compared to normal horses, concluding that IL-31 is involved in IBH pathology. The same group then vaccinated IBH horses with an IL-31 vaccine and claimed to alleviate clinical scores in IBH affected horses (Olomski et al. Allergy 2020; 75(4): 862-871).

It would be desirable to provide for alternative approaches to treat pruritic and allergic disorders in equine mammals, such as horses. In order to determine if IL-31 plays a key role in equine pruritus, a key clinical sign of allergic skin disease in horses, it would be desirable to establish an IL-31 induced pruritus model in horses. Such a model could be used as a surrogate for naturally occurring IL-31 mediated clinical disease. This would enable the assessment of the effectiveness of candidate horse IL-31 inhibitors in reducing pruritic behavior in the treated horses and alleviating IL-31-mediated disorders affecting equine mammals.

### SUMMARY OF THE INVENTION

The present invention provides an IL-31 horse pruritus model comprising administering equine IL-31 to horses to produce a pruritic response; quantitatively measuring pruritic responses in the horses which were administered equine IL-31; administering a candidate horse IL-31 inhibitor; and assessing the effectiveness of the candidate horse IL-31 inhibitor in reducing pruritic behavior in the treated horses by challenging the horses with equine IL-31 following the administration of the candidate horse IL-31 inhibitor.

In one embodiment, the equine IL-31 administered to the horses to produce the pruritic response is recombinant equine IL-31. In one embodiment, the recombinant equine IL-31 is encoded by a nucleic acid comprising the following nucleotide sequence (5′ to 3′):

(SEQ ID NO: 1)
ATGGGCTGGTCCTGCATCATTCTGTTTCTGGTGGCCACAGCCACCGGCGT
GCACTCTGGACCTATCTATCAGCTGCAGCCCAAAGAGATCCAGGCCATCA
TCGTGGAACTGCAGAACCTGAGCAAGAAGCTGCTGGACGACTACCTGAAC
AAAGAAAAGGGCGTGCAGAAGTTCGACAGCGACCTGCCTAGCTGCTTCAC
CAGCGATTCTCAGGCCCCTGGCAACATCAACAGCAGCGCCATCCTGCCTT
ACTTCAAGGCCATCTCTCCCAGCCTGAACAACGACAAGAGCCTGTACATC
ATCGAGCAGCTGGACAAGCTGAACTTCCAGAACGCCCCTGAAACCGAGGT
GTCCATGCCTACCGACAACTTCGAGCGGAAGCGGTTCATCCTGACCATCC
TGCGGTGGTTCAGCAACTGCCTGGAACACAGAGCCCAGCACCACCACCAT
CACCATTGATAAGCTT.

In one embodiment, the equine IL-31 is administered parenterally or intradermally to the horse. Parenteral routes can include subcutaneous, intramuscular, and intravenous routes, for example.

In a further embodiment, the equine IL-31 is administered to the horse at a dose of 0.05 to 1 μg/kg. In another embodiment, the equine IL-31 is administered to the horse at a dose of 0.1 to 0.25 μg/kg.

In yet another embodiment, the pruritic response is a transient response. In one embodiment, the transient pruritic response lasts less than 24 hours. In a further embodiment, the pruritic responses in the horses induced by equine IL-31 are selected from; biting or scratching at self, rubbing against objects, feet stomping, tail flicking, head or body shaking, rolling, twitching of skin, and combinations thereof.

In one embodiment, pruritic behavior measurements are performed using real-time surveillance or video recording using a categorical scoring system, or by timing pruritic events throughout an observation window. In one embodiment, at consecutive time intervals, “yes/no” decisions are made as to whether pruritic behavior was displayed by each horse. In a specific embodiment, the consecutive time intervals are 1 minute intervals. In another embodiment, at the end of a designated observation period, the numbers of yes determinations are added together to come up with a cumulative Pruritic Score (PS).

In one embodiment, a first PS measurement is a baseline score measured immediately prior to the equine IL-31 challenge. In another embodiment, an additional PS measurement is determined following the equine IL-31 challenge.

In another embodiment, a timer can be used to count in seconds how long pruritic events are occurring during the observation window. In this embodiment, an observer will either use real-time surveillance or watch a video recording and start a timer when they observe pruritic behaviors, and then stop the timer when the pruritic behavior stops. This is repeated throughout the observation window to obtain a score of “x” seconds of pruritus per observation window.

In one embodiment, the candidate horse IL-31 inhibitor comprises a small molecule compound, an anti-IL31 antibody, or an IL-31 vaccine mimotope.

In one embodiment, the candidate horse IL-31 inhibitor comprises a small molecule compound. In a specific embodiment, the small molecule compound is an inhibitor of the janus kinase pathway, the mitogen activated protein kinase pathway, or the Akt/protein kinase B pathway. In one embodiment, the small molecule compound is combined with a pharmaceutically acceptable carrier. For example, the small molecule compound can be mixed in food and/or water, or delivered in admixture with a suitable carrier, diluent, or excipient such as sterile water, physiological saline, glucose, or the like. In one embodiment, the carrier is a feed carrier.

In a further embodiment, the candidate horse IL-31 inhibitor comprises an IL-31 vaccine mimotope. In one embodiment, the IL-31 vaccine mimotope is an equine IL-31 mimotope, a feline IL-31 mimotope, or a canine IL-31 mimotope.

In another embodiment, the IL-31 vaccine mimotope is a constrained mimotope or a linear mimotope. In one embodiment, the constrained mimotope is a chemically-linked cyclic peptide.

In one embodiment, the equine IL-31 mimotope is based on the IL-31 BC helix. In one embodiment, the equine BC helix mimotope comprises the amino acid sequence NSSAILPYFKAISPSLNNDKSLYIIEQLDKLNF (SEQ ID NO: 2) or a variant thereof that retains anti-IL-31 binding.

In another embodiment, the equine IL-31 mimotope is based on the epitope to which a monoclonal (mAb) antibody designated 15H05 or 1505 specifically binds. In one embodiment, the equine 1505 mimotope comprises the amino acid sequence TEVSMPTDNFERKRFILTC (SEQ ID NO:4) or a variant thereof that retains anti-IL-31 binding.

In yet another embodiment, the equine IL-31 mimotope is based on the IL-31 A helix. In one embodiment, the equine A helix mimotope comprises the amino acid sequence GPIYQLQPKEIQAIIVELQNLSKK (SEQ ID NO: 6) or a variant thereof that retains anti-IL-31 binding.

In a further embodiment, the equine IL-31 mimotope is based on the IL-31 AB loop. In one embodiment, the equine AB loop mimotope comprises the amino acid sequence KEKGVQKFDS (SEQ ID NO: 7) or a variant thereof that retains anti-IL-31 binding.

In another embodiment, the feline IL-31 mimotope is based on the IL-31 1505 mAb epitope. In one embodiment, the feline 1505 mAb mimotope comprises the amino acid sequence AKVSMPADNFERKNFILT (SEQ ID NO: 3) or a variant thereof that retains anti-IL-31 binding.

In yet another embodiment, the canine IL-31 mimotope is based on the IL-31 1505 mAb epitope. In one embodiment, the canine 1505 mimotope comprises the amino acid sequence TEISVPADTFECKSFILT (SEQ ID NO: 5) or a variant thereof that retains anti-IL-31 binding.

In one embodiment, the mimotope is a constrained mimotope or a linear mimotope. In a particular embodiment, the constrained mimotope is a chemically-linked cyclic peptide.

In some embodiments, the mimotope is combined with a carrier polypeptide. In one embodiment, the mimotope is chemically conjugated to the carrier polypeptide. In other embodiments, the carrier polypeptide and the mimotope are part of a recombinant fusion protein.

In one embodiment, the carrier polypeptide includes a bacterial toxoid or a derivative thereof, keyhole limpet hemocyanin (KLH), a virus-like particle, or combinations thereof. In one embodiment, the bacterial toxoid or derivative is selected from tetanus toxoid, a diphtheria toxoid, a tetanus toxoid, the outer membrane protein complex from group B N. meningitidis, Pseudomonas exotoxin, or the nontoxic mutant of diphtheria toxin (CRM197). In another embodiment, the virus-like particle is selected from HBsAg, HBcAg, E. coli bacteriophage Qbeta, Norwalk virus, canine distemper virus (CDV), influenza HA, or cucumber mosaic virus (CuMV). In a specific embodiment, the mimotope is combined with a carrier polypeptide which comprises or consists of CRM197.

In one embodiment, the adjuvant is selected from an oil-in-water adjuvant, a polymer and water adjuvant, a water-in-oil adjuvant, an aluminum hydroxide adjuvant, a vitamin E adjuvant or combinations thereof.

In one embodiment, the adjuvant is a formulation comprising a saponin, a sterol, a quaternary ammonium compound, and a polymer. In a specific embodiment, the saponin is Quil A or a purified fraction thereof, the sterol is cholesterol, the quaternary ammonium compound is dimethyl dioctadecyl ammonium bromide (DDA), and the polymer is polyacrylic acid.

In another embodiment, the adjuvant comprises the combination of one or more isolated immunostimulatory oligonucleotides, a sterol, and a saponin. In a specific embodiment, the one or more isolated immunostimulatory oligonucleotides comprises CpG, the sterol is cholesterol, and the saponin is Quil A or a purified fraction thereof.

In one embodiment, the IL-31 vaccine mimotope is combined with an adjuvant comprising an oil-in-water adjuvant containing one or more immunostimulatory oligonucleotides.

In another embodiment, the candidate IL-31 vaccine mimotope is present as part of a vaccine composition together with a carrier polypeptide and an adjuvant or adjuvant formulation, as described herein.

In one embodiment, the candidate horse IL-31 inhibitor comprises an isolated IL-31 antibody or antigen-binding portion thereof. In one embodiment, the candidate horse IL-31 inhibitor is an equinized or fully equine monoclonal antibody. In another embodiment, the equinized or fully equine monoclonal antibody binds to an epitope on IL-31 that is equivalent to one of the IL-31 vaccine mimotopes described herein.

In a further embodiment, the candidate horse IL-31 inhibitor is administered parenterally. Parenteral routes can include subcutaneous, intramuscular, and intravenous routes, for example. In another embodiment, the candidate horse IL-31 inhibitor is administered orally.

The present invention further provides the use of the IL-31 horse pruritus model described herein to identify a small molecule IL-31 inhibitor/mediator, a neutralizing monoclonal antibody raised against equine IL-31, or an IL-31 vaccine mimotope composition as being capable of protecting a horse against an IL-31 mediated disorder. In one embodiment, the neutralizing monoclonal antibody targets the IL-31 1505 mAb epitope.

The present invention also provides a method of protecting a horse against an IL-31 mediated disorder. Such a method includes administering to the horse a therapeutically effective amount of a small molecule IL-31 inhibitor/mediator or an IL-31 vaccine mimotope composition identified in the examples herein as being capable of protecting a horse against an IL-31 mediated disorder.

In one embodiment, the IL-31-mediated disorder is a pruritic and/or allergic condition. In some embodiments, the pruritic condition is due to allergic reactions due to ectoparasites or environmental allergens, food allergies, atopic dermatitis, urticaria, or skin infections from staphylococcus or fungus. In one embodiment, the pruritic and/or allergic condition is pruritic allergic dermatitis. Insect-bite hypersensitivity and atopic dermatitis are each forms of pruritic allergic dermatitis. Other examples of pruritic disorders can include eczema, psoriasis, scleroderma, and pruritus. Also, allergic conditions can include allergic dermatitis, summer eczema, urticaria, heaves, inflammatory airway disease, recurrent airway obstruction, airway hyper-responsiveness, chronic obstruction pulmonary disease, and inflammatory processes resulting from autoimmunity. In other embodiments, the IL-31 mediated disorder is tumor progression. In some embodiments, the IL-31 mediated disorder is eosinophilic disease or mastocytomas.

### BRIEF DESCRIPTION OF THE SEQUENCES

SEQ ID NO:1 is a DNA sequence used to express an equine IL-31 used to elicit pruritic responses in horses.

SEQ ID NO:2 is an amino acid sequence of an equine IL-31 mimotope comprised within a peptide with code ZTS-765 based on the IL-31 BC helix.

SEQ ID NO:3 is an amino acid sequence of a feline IL-31 mimotope comprised within a peptide with code ZTS-422 based on the IL-31 1505 mAb epitope.

SEQ ID NO:4 is an amino acid sequence of an equine IL-31 mimotope comprised within peptides with codes ZTS-7240 and ZTS-418 based on the IL-31 1505 mAb epitope.

SEQ ID NO:5 is an amino acid sequence of a canine IL-31 mimotope comprised within a peptide with code ZTS-564 based on the IL-31 1505 mAb epitope.

SEQ ID NO:6 is an amino acid sequence of an equine IL-31 mimotope comprised within a peptide with code ZTS-7241 based on the IL-31 A helix.

SEQ ID NO:7 is an amino acid sequence of an equine IL-31 mimotope comprised within a peptide with code ZTS-7242 based on the IL-31 AB loop.

SEQ ID NO: 8 is the amino acid sequence of a feline IL-31 wildtype sequence designated herein as Feline_IL31_wildtype.

SEQ ID NO: 9 is the amino acid sequence of a canine IL-31 sequence designated herein as Canine_IL31.

SEQ ID NO: 10 is the amino acid sequence of an equine IL-31 polypeptide (Equine_IL31 polypeptide).

SEQ ID NO: 11 is the amino acid sequence of an alternative version of an equine IL-31 polypeptide (Equine_IL31-alternative polypeptide version).

### BRIEF DESCRIPTION OF THE DRAWINGS

FIG. 1: Summary graph of average baseline percent pruritus scores to average post challenge percent pruritus scores for each of the first 5 treatment groups. 1.0 μg/kg (N=3), 0.5 μg/kg (N=7), 0.25 μg/kg (N=7), 0.1 μg/kg (N=8), 0.05 μg/kg (N=8)

FIG. 2. IL-31 Homology Model Using IL-6 Structure and the Equine IL-31 Mimotopes Identified for Evaluation

FIG. 3. First Equine IL-31 Mimotope Study Serology Data

FIG. 4. Second Equine IL-31 Mimotope Study Serology Data

### DEFINITIONS

Before describing the present invention in detail, several terms used in the context of the present invention will be defined. In addition to these terms, others are defined elsewhere in the specification, as necessary. Unless otherwise expressly defined herein, terms of art used in this specification will have their art-recognized meanings

As used in the specification and claims, the singular form “a”, “an” and “the” include plural references unless the context clearly dictates otherwise. For example, reference to “an antibody” includes a plurality of such antibodies. As another example, reference to “a mimotope”, “an IL-31 mimotope” and the like includes a plurality of such mimotopes.

As used herein, the term “comprising” is intended to mean that the compositions and methods include the recited elements, but not excluding others.

As used herein, the term “vaccine composition” includes at least one antigen or immunogen in a pharmaceutically acceptable vehicle useful for inducing an immune response in a host. Vaccine compositions can be administered in dosages, and by techniques well known to those skilled in the medical or veterinary arts, taking into consideration factors such as the age, sex, weight, species and condition of the recipient mammal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal). Vaccine compositions can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences” (1990), may be consulted to prepare suitable preparations, without undue experimentation.

The term “immune response” as used herein refers to a response elicited in an animal or human. An immune response may refer to cellular immunity (CMI), humoral immunity, or may involve both. The present invention also contemplates a response limited to a part of the immune system. Usually, an “immunological response” includes, but is not limited to, one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response, such that resistance to the disease or disorder will be enhanced, and/or the clinical severity of the disease reduced. Such protection will be demonstrated by either a reduction or lack of symptoms normally displayed by an affected host, a quicker recovery time, and/or a lowered antigen (e.g., IL-31) titer in the affected host.

The term “protecting”, “protect” and the like as used herein means conferring a therapeutic immunological response to a host mammal, such that resistance to a disease or disorder will be enhanced, and/or the clinical severity of the disease reduced in the host mammal.

As used herein, the term “immunogenicity” means capable of producing an immune response in a host mammal against an antigen or antigens. This immune response forms the basis of the protective immunity elicited by a vaccine against a specific antigen.

As used herein, immunizing, immunization, and the like is the process whereby a mammal is made immune or resistant to a disease, typically by the administration of a vaccine. Vaccines stimulate the mammal's own immune system to protect the mammal against subsequent disease.

An “adjuvant” as used herein means a composition comprised of one or more substances that enhances the immune response to an antigen(s). The mechanism of how an adjuvant operates is not entirely known. Some adjuvants are believed to enhance the immune response by slowly releasing the antigen, while other adjuvants are strongly immunogenic in their own right, and are believed to function synergistically.

Epitope, as used herein, refers to the antigenic determinant recognized by the CDRs of the antibody. In other words, epitope refers to that portion of any molecule capable of being recognized by, and bound by, an antibody. Unless indicated otherwise, the term “epitope” as used herein, refers to the region of IL-31 to which an anti-IL-31 agent is reactive to.

An “antigen” is a molecule or a portion of a molecule capable of being bound by an antibody which is additionally capable of being recognized by, and bound by, an antibody (the corresponding antibody binding region may be referred to as a paratope). In general, epitopes consist of chemically active surface groupings of molecules, for example, amino acids or sugar side chains, and have specific three-dimensional structural characteristics as well as specific charge characteristics. Epitopes are the antigenic determinant on a protein that is recognized by the immune system. The components of the immune system recognizing epitopes are antibodies, T-cells, and B-cells. T-cell epitopes are displayed on the surface of antigen-presenting cells (APCs) and are typically 8-11 (MHC class I) or 15 plus (MHC class II) amino acids in length. Recognition of the displayed MHC-peptide complex by T-cells is critical to their activation. These mechanisms allow for the appropriate recognition of self versus “non-self” proteins such as bacteria and viruses. Independent amino acid residues that are not necessarily contiguous contribute to interactions with the APC binding cleft and subsequent recognition by the T-Cell receptor (Janeway, Travers, Walport, Immunobiology: The Immune System in Health and Disease. 5th edition New York: Garland Science; 2001). Epitopes that are recognized by soluble antibodies and cell surface associated B-cell receptors vary greatly in length and degree of continuity (Sivalingam and Shepherd, Immunol. 2012 July; 51(3-4):304-309 9). Again even linear epitopes or epitopes found in a continuous stretch of protein sequence will often have discontiguous amino acids that represent the key points of contact with the antibody paratopes or B-cell receptor. Epitopes recognized by antibodies and B-cells can be conformational with amino acids comprising a common area of contact on the protein in three dimensional space and are dependent on tertiary and quaternary structural features of the protein. These residues are often found in spatially distinct areas of the primary amino acid sequence.

A “mimotope” as used herein is a linear or constrained peptide which mimics an antigen's epitope. A mimotope may have a primary amino acid sequence capable of eliciting a T-cell effector response and/or a three dimensional structure necessary to bind B-cells resulting in maturation of an acquired immunological response in an animal. In one embodiment, an antibody for a given epitope antigen will recognize a mimotope which mimics that epitope. An IL-31 mimotope may alternatively be referred to herein as an IL-31 peptide mimotope. In some embodiments, a mimotope (linear or constrained) for use in the compositions and/or methods of the present invention is and/or comprises as part thereof a peptide which is from about 5 amino acid residues to about 40 amino acid residues in length, such as about 5 to about 10, 10 to about 20, 20 to about 30, or 30 to about 40 amino acid residues in length. In some embodiments, the peptide mimotope is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues in length. It is to be understood that the peptide mimotopes may comprise some amino acid residues which facilitate chemical conjugation, such as terminal Cysteines, may comprise linkers, or chemical groups such as n-terminal acetyl or c-terminal amide groups. The mimotopes are included in a vaccine composition which can further include a carrier polypeptide and/or an adjuvant or adjuvant mixture.

The term “variant” as used herein refers to a peptide, polypeptide or a nucleic acid sequence encoding a peptide or polypeptide, that has or encodes one or more conservative amino acid variations or other minor modifications such that the corresponding peptide or polypeptide has substantially equivalent function when compared to the wild-type peptide or polypeptide. Ordinarily, variant peptide mimotopes for use in the experimental model disclosed herein will have at least 30% identity to the parent mimotopes described herein, more preferably at least 50%, more preferably at least 75%, more preferably at least 80%, more preferably at least 85%, more preferably at least 90%, and most preferably at least 95% sequence identity to the parent mimotope. Such variant mimotopes retain anti-IL-31 binding. Also, typically a variant equine IL-31 for use in the experimental model disclosed herein will preferably have at least 50%, more preferably at least 75%, more preferably at least 80%, more preferably at least 85%, more preferably at least 90%, and most preferably at least 95% sequence identity to the wild-type mature equine IL-31. It is understood that the equine IL-31 variant retains the ability to induce itch in the horses to which the antigen is administered. Also, the equine IL-31 may include tags or labels to facilitate its protein purification and/or its recovery. Furthermore, the nucleic acid sequence encoding the equine IL-31 may be codon-optimized to increase protein production, if desired.

The term “specifically” in the context of antibody binding, refers to high avidity and/or high affinity binding of an antibody to a specific antigen, i.e., a polypeptide, or epitope. In many embodiments, the specific antigen is an antigen (or a fragment or subfraction of an antigen) used to immunize the animal host from which the antibody-producing cells were isolated. Antibody specifically binding an antigen is stronger than binding of the same antibody to other antigens. Antibodies which bind specifically to a polypeptide may be capable of binding other polypeptides at a weak, yet detectable level (e.g., 10% or less of the binding shown to the polypeptide of interest). Such weak binding, or background binding, is readily discernible from the specific antibody binding to a subject polypeptide, e.g. by use of appropriate controls. In general, specific antibodies bind to an antigen with a binding affinity with a K_{D} of 10⁻⁷M or less, e.g., 10⁻⁸M or less (e.g., 10⁻⁹ M or less, 10⁻¹⁰ or less, 10⁻¹¹ or less, 10⁻¹² or less, or 10⁻¹³ or less, etc.).

As used herein, the term “antibody” refers to an intact immunoglobulin having two light and two heavy chains. Thus a single isolated antibody or fragment may be a polyclonal antibody, a monoclonal antibody, a synthetic antibody, a recombinant antibody, a chimeric antibody, a heterochimeric antibody, a caninized antibody, a felinized antibody, an equinized antibody, a fully canine antibody, a fully feline antibody, a fully equine antibody, or a fully human antibody. The term “antibody” preferably refers to monoclonal antibodies and fragments thereof (e.g., including but not limited to, antigen-binding portions of the antibody), and immunologic binding equivalents thereof that can specifically bind to the IL-31 protein and fragments or modified fragments thereof. Such fragments and modified fragments of IL-31 can include the IL-31 peptide mimotopes employed in the various embodiments of this invention. For example, an antibody for a given epitope on IL-31 will recognize an IL-31 peptide mimotope which mimics that epitope. The term antibody is used both to refer to a homogeneous molecular, or a mixture such as a serum product made up of a plurality of different molecular entities.

“Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

“Monoclonal antibody” or mAb as defined herein is an antibody produced by a single clone of cells (e.g., a single clone of hybridoma cells) and therefore a single pure homogeneous type of antibody. All monoclonal antibodies produced from the same clone are identical and have the same antigen specificity. The term “monoclonal” pertains to a single clone of cells, a single cell, and the progeny of that cell.

“Fully equine antibody” as defined herein is a monoclonal antibody produced by a clone of cells (typically a CHO cell line) and therefore a single pure homogeneous type of antibody. Antibodies identified from single B cells of immunized mammals, such as dogs are created as recombinant IgG proteins following identification of their variable domain sequences. Grafting of these variable domains onto equine constant domains (heavy chain and light chain kappa or lambda constant) results in the generation of recombinant fully equine antibodies. All fully equine monoclonal antibodies produced from the same clone are identical and have the same antigen specificity. The term “monoclonal” pertains to a single clone of cells, a single cell, and the progeny of that cell. “Fully Equine” antibodies are genetically engineered antibodies that contain no sequence derived from non-equine immunoglobulin. Fully equine antibodies are equine immunoglobulin sequences (recipient antibody) in which hypervariable region residues are derived from a naturally occurring equine antibody (donor antibody) having the desired specificity, affinity, and capacity. Furthermore, fully equine antibodies may include residues that are not found in the recipient antibody or in the donor antibody, such as including, but not limited to changes in the CDRs to modify affinity. These modifications are made to further refine antibody performance. In general, the fully equine antibody will include substantially all of at least one, and typically two, variable domains, in which all or substantially all of the hypervariable regions correspond to those of a equine immunoglobulin sequence and all or substantially all of the FRs are those of an equine immunoglobulin sequence. The fully equine antibody optionally also will comprise a complete, or at least a portion of an immunoglobulin constant region (Fc), typically that of equine immunoglobulin sequence.

“Equinized” forms of non-equine (e.g., murine) antibodies are genetically engineered antibodies that contain minimal sequence derived from non-equine immunoglobulin. Equinized antibodies are equine immunoglobulin sequences (recipient antibody) in which hypervariable region residues of the recipient are replaced by hypervariable region residues from a non-equine species (donor antibody) such as mouse having the desired specificity, affinity, and capacity. In some instances, framework region (FR) residues of the equine immunoglobulin sequences are replaced by corresponding non-equine residues. Furthermore, equinized antibodies may include residues that are not found in the recipient antibody or in the donor antibody. These modifications are made to further refine antibody performance. In general, the equinized antibody will include substantially all of at least one, and typically two, variable domains, in which all or substantially all of the hypervariable regions correspond to those of a non-equine immunoglobulin sequence and all or substantially all of the FRs are those of an equine immunoglobulin sequence. The equinized antibody optionally also will comprise a complete, or at least a portion of an immunoglobulin constant region (Fc), typically that of an equine immunoglobulin sequence.

The term “antigen binding region”, “antigen-binding portion”, and the like as used throughout the specification and claims refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues.

The term “isolated” means that the material (e.g., antibody) is separated and/or recovered from a component of its natural environment. Contaminant components of its natural environment are materials that would interfere with diagnostic or therapeutic uses for the material, and may include enzymes, hormones, and other proteinaceous or nonproteinaceous solutes. In preferred embodiments, the material will be purified to greater than 95% by weight of the material, and most preferably more than 99% by weight. Isolated material includes the material in situ within recombinant cells since at least one component of the material's natural environment will not be present. Ordinarily, however, isolated material will be prepared by at least one purification step.

A “subject” or “patient” refers to a mammal in need of treatment that can be affected by molecules of the invention. Mammals that can be treated in accordance with the invention include equine mammals, such as horses, donkeys, and zebras, with horses being particularly preferred examples.

A “therapeutically effective amount” (or “effective amount”) refers to an amount of an active ingredient, e.g., an agent according to the invention, sufficient to effect beneficial or desired results when administered to a subject or patient. An effective amount can be administered in one or more administrations, applications or dosages. A therapeutically effective amount of a composition according to the invention may be readily determined by one of ordinary skill in the art. In the context of this invention, a “therapeutically effective amount” is one that produces an objectively measured change in one or more parameters associated with treatment of an IL-31 mediated disorder, such as a pruritic condition or an allergic condition, or tumor progression, including clinical improvement in symptoms. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art.

As used herein, the term “therapeutic” encompasses the full spectrum of treatments for a disease or disorder. A “therapeutic” agent of the invention may act in a manner that is prophylactic or preventive, including those that incorporate procedures designed to target animals that can be identified as being at risk (pharmacogenetics); or in a manner that is ameliorative or curative in nature; or may act to slow the rate or extent of the progression of at least one symptom of a disease or disorder being treated.

“Treatment”, “treating”, and the like refers to both therapeutic treatment and prophylactic or preventative measures. Animals in need of treatment include those already with the disorder as well as those in which the disorder is to be prevented. The term “treatment” or “treating” of a disease or disorder includes preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

The term “allergic condition” is defined herein as a disorder or disease caused by an interaction between the immune system and a substance foreign to the body. This foreign substance is termed “an allergen”. Common allergens include aeroallergens, such as pollens, dust, molds, dust mite proteins, injected saliva from insect bites, etc. Examples of allergic conditions include, but are not limited to, the following: allergic dermatitis, summer eczema, urticaria, heaves, inflammatory airway disease, recurrent airway obstruction, airway hyper-responsiveness, chronic obstructive pulmonary disease, and inflammatory processes resulting from autoimmunity, such as Irritable bowel syndrome (IBS). Allergic dermatitis in horses encompasses many different entities or syndromes including insect-bite hypersensitivity (IBH), atopic dermatitis (AD), food allergy and urticaria. IBH is the most common form of allergic dermatitis in horses worldwide.

The term “pruritic condition” is defined herein as a disease or disorder characterized by an intense itching sensation that produces the urge to rub or scratch the skin to obtain relief. Examples of pruritic conditions include, but are not limited to the following: atopic dermatitis, allergic dermatitis, eczema, psoriasis, scleroderma, and pruritus. Allergic dermatitis in horses encompasses many different entities or syndromes including insect-bite hypersensitivity (IBH), atopic dermatitis (AD), food allergy and urticaria.

A “composition” is intended to mean a combination of active agent and another compound or composition which can be inert (e.g., a label), or active, such as an adjuvant.

The phrase “pharmaceutically acceptable carrier” as used herein means a pharmaceutically acceptable material, composition or vehicle, such as a liquid or solid filler, diluent, excipient, solvent or encapsulating material. In the case of a vaccine composition, the terms “pharmaceutically acceptable carrier” and “pharmaceutically acceptable vehicle” are interchangeable, and refer to a fluid vehicle for containing vaccine antigens that can be injected into a host without adverse effects. Pharmaceutically acceptable carriers suitable for use in the invention are well known to those of skill in the art. Such carriers include, without limitation, water, saline, buffered saline, phosphate buffer, alcoholic/aqueous solutions, emulsions or suspensions. Other conventionally employed diluents, adjuvants and excipients, may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, e.g., citrates, ascorbic acid, gluconic acid, histidine-HCl, carbonic acid, tartaric acid, succinic acid, acetic acid, or phthalic acid, Tris, trimethanmine hydrochloride, or phosphate buffers. Parenteral carriers can include sodium chloride solution, Ringer's dextrose, dextrose, trehalose, sucrose, and sodium chloride, lactated Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents (e.g., EDTA), inert gases and the like may also be provided in the pharmaceutical carriers. The present invention is not limited by the selection of the carrier. The preparation of these pharmaceutically acceptable compositions, from the above-described components, having appropriate pH isotonicity, stability and other conventional characteristics is within the skill of the art. See, e.g., texts such as Remington: The Science and Practice of Pharmacy, 20th ed, Lippincott Williams & Wilkins, publ., 2000; and The Handbook of Pharmaceutical Excipients, 4.sup.th edit., eds. R. C. Rowe et al, APhA Publications, 2003.

### DETAILED DESCRIPTION OF THE INVENTION

An objective of the present invention was to develop an experimental model to test whether equine IL-31 induces pruritus in horses which were administered the equine IL-31 and if so, to test whether candidate horse IL-31 inhibitors can block or inhibit pruritic behaviors which had been induced in the horses. Through this model, it was established that IL-31 plays a major role in equine pruritus, which is a key clinical sign of allergic skin disease in horses. It was also established that treatment with various test compounds could inhibit the pruritic behaviors in the horses.

The IL-31 induced pruritus model was developed in horses, as a surrogate for naturally occurring clinical disease. In the developed equine model, to determine whether IL-31 could induce pruritic behaviors in horses, equine bioactive IL-31 was cloned, expressed, and purified. Varying levels of IL-31 were then injected intravenously into horses, and pruritic behaviors of horses were observed.

The IL-31 horse pruritus model of the present invention includes administering equine IL-31 to horses to produce a pruritic response; quantitatively measuring pruritic responses in the horses which were administered equine IL-31; administering a candidate horse IL-31 inhibitor; and assessing the effectiveness of the candidate horse IL-31 inhibitor in reducing pruritic behavior in the treated horses by challenging the horses with equine IL-31 following the administration of the candidate horse IL-31 inhibitor.

In one embodiment of the model, the equine IL-31 is recombinant equine IL-31 polypeptide corresponding to the mature wild-type equine IL-31 protein, such as from any of the various known Equus species, such as Equus caballus, Equus przewalski, or Equus asinus. However, variants of the wild-type mature equine IL-31 protein are also contemplated provided they can induce itch in the horse. For example, the variant equine IL-31 can include labels or tags, such as histidine tags designed to facilitate protein purification and/or recovery. Also, it is anticipated that the mature equine IL-31 protein need not be full length and/or may include minor modifications relative to the wild-type sequence, such as conservative substitutions, for example. In a specific embodiment, the recombinant equine IL-31 is encoded by a nucleic acid comprising the nucleotide sequence of SEQ ID NO:1, although the model is not limited as such. SEQ ID NO: 1 encodes the following amino acid sequence, wherein the predicted signal sequence is underlined:

(SEQ ID NO: 10)
MGWSCIILFLVATATGVHSGPIYQLQPKEIQAIIVELQNLSKKLLDDYLN
KEKGVQKFDSDLPSCFTSDSQAPGNINSSAILPYFKAISPSLNNDKSLYI
IEQLDKLNFQNAPETEVSMPTDNFERKRFILTILRWFSNCLEHRAQHHHH
HH.

The sequence below represents an alternative version of an equine IL-31 amino acid sequence, wherein the predicted signal sequence is underlined.

(SEQ ID NO: 11)
MVSHIGTTAFALFLLCCLGTLMFSHTGPIYQLQPKEIQAIIVELQNLSKK
LLDDYLNKEKGVQKFDSDLPSCFTSDSQAPGNINSSAILPYFKAISPSLN
NDKSLYIIEQLDKLNFQNAPETEVSMPTDNFERKRFILTILRWFSNCLEH
HHHHH.

However, the invention is not limited to SEQ ID NO: 10 or SEQ ID NO: 11. It is understood that the signal sequence is usually removed in the mature protein. In one embodiment the equine IL-31 is administered parenterally, such as subcutaneously, intramuscularly, or intravenously. In another embodiment, the equine IL-31 is administered intradermally.

In the model exemplified herein, five concentrations of IL-31 were used to induce pruritus in horses. These doses were 1 μg/kg, 0.5 μg/kg, 0.25 μg/kg, 0.1 μg/kg and 0.05 μg/kg. Thus, according to one embodiment, the equine IL-31 is administered at a dose of 0.05 to 1 μg/kg. In another embodiment, the equine IL-31 is administered at a dose of 0.1 to 0.25 μg/kg.

The goal of this model is to induce a strong pruritic response without having too high of concentrations that a therapeutic cannot overcome or too strong of a pruritic response that the animals are uncomfortable.

In this study, it was determined that 0.25 μg/kg dose of IL-31 elicited an appropriate pruritic response. However, the model is not limited to this specific dose of equine IL-31.

The current developed horse IL-31 induced itch model was developed by evaluating the ability of different doses of equine IL-31 to induce a pruritic response, as described herein. Once established and characterized, additional in vivo studies described in the example section have investigated the ability of candidate horse IL-31 inhibitors, such as, but not limited to, IL-31 mimotope vaccines and small molecule compounds, such as a janus kinase inhibitor (oclacitnib maleate), to reduce the pruritic response elicited by equine IL-31. Animal models are an essential component in the discovery of new therapeutics and sustain the evaluation of candidate therapeutics during the early stages drug development. Here, the current invention, has been shown to be a reliable surrogate model for horse IL-31 induced diseases as demonstrated by the efficacy of the test compounds used to evaluate the validity of the developed model.

In some embodiments, the pruritic response in the horses which were administered the equine IL-31 is a transient response, such as but not limited to, a transient response lasting less than 24 hours.

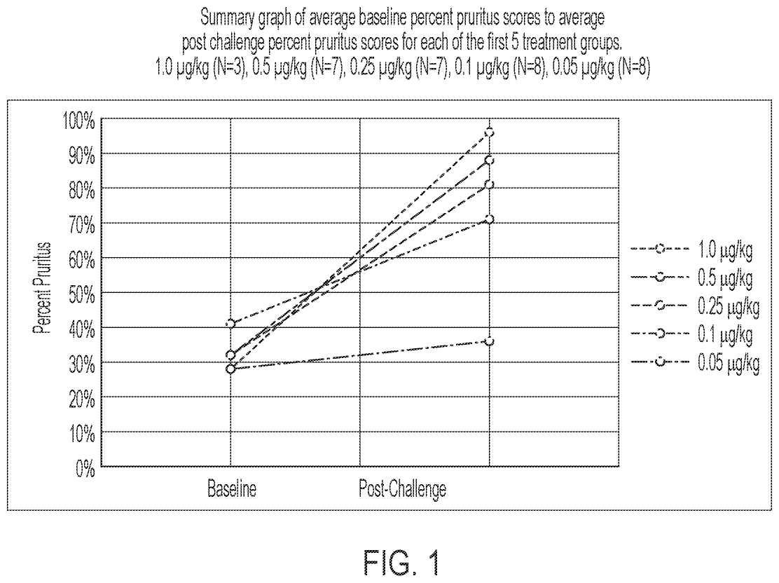

Observations of normal pruritic behavior (baseline pruritus scores) were made prior to the administration of IL-31 challenge. As described in Example 1, the horses were scored the same way as post challenge observations except no IL-31 challenge was administered and observation of normal pruritic behavior was made for 30 minutes. Following baseline pruritus score, horses were administered the IL-31 challenge. IL-31 challenge was administered intravenously to each animal via a jugular vein. The horse ID and time of administration was recorded. Recording of pruritic activity began approximately 15 to 25 minutes after the last of the horses is administered the IL-31 challenge (Table 2 and FIG. 1).

In order to characterize pruritic behavior, horses were observed for any behavior that can be identified as pruritus. Classical signs of pruritus in horses include the following: biting or scratching at self, rubbing against objects, feet stomping, tail flicking, head or body shaking, rolling, twitching of skin and any combination thereof.

In one embodiment, the pruritic behavior measurements are performed using real-time surveillance or video recording using a categorical scoring system, or by timing pruritic events throughout an observation window. For example, in one embodiment, at consecutive time intervals, “yes/no” decisions are made as to whether pruritic behavior is being displayed by each horse. In one embodiment, a “yes” response to pruritic behavior is indicated by marking a “1” and a “no” response is indicated by marking a “0” during the 1-minute interval. In one embodiment, the cumulative number of yes responses over the designated observation period are added to determine a cumulative pruritus score (PS) for each horse. In one embodiment, the observation period is 120 minutes. In one embodiment, a baseline pruritus score (first PS measurement) is measured immediately prior to the equine IL-31 challenge. In another embodiment, an additional PS measurement is determined following the equine IL-31 challenge.

The cytokine IL-31 has been implicated in pruritus in certain subjects/species, such as mice, dogs, cats, humans and monkeys, and has therefore been used to build models of pruritus associated with allergic dermatitis in some of these species. This cytokine is secreted by CD4⁺ T cells, and when bound to its receptor, activates a number of pathways including those in peripheral nerves to induce pruritic behavior.

The equine model developed by the present inventors will serve as a proof of concept model that can be used to determine dose and efficacy for a potential anti-pruritic drug (e.g. small molecule, isolated monoclonal antibody or IL-31 mimetic vaccine).

As described in Example 1, of the five doses of IL-31 tested, it was determined that 0.25 μg/kg was a preferred dose, although the present invention is not limited to this dose. On average, 0.25 μg/kg increased pruritus from 32% at baseline to 81% post challenge. The 1 μg/kg dose was not a preferred option due to the need for diphenhydramine to make the horses more comfortable after the post challenge observations. In the 0.5 μg/kg group, there were still some horses that had post challenge pruritus scores of 100% therefore this dose was also not optimal. The 0.1 μg/kg dose had a reasonable pruritic response with an average baseline pruritic score of 41% and an average post challenge pruritic score of 71% but it was not as robust of a response as the 0.25 μg/kg dose. The 0.05 μg/kg did not elicit a strong pruritic response with only an average post challenge pruritic score of 48%. The skilled person will understand that equine IL-31 concentrations ranging from 0.05 μg/kg to 1 μg/kg are useful for eliciting a pruritic response, although concentrations of equine IL-3 of about 0.1 to about 0.25 to about μg/kg may be preferred in some embodiments.

With the establishment of a suitable IL-31 induced horse itch model, utility was demonstrated by evaluating the ability of IL-31 inhibitors to reduce pruritis once induced by equine IL-31. In the example section, two types of IL-31 inhibitors were evaluated; IL-31 mimotope vaccines and a small molecule compound, which is a known janus kinase inhibitor called oclacitinib maleate.

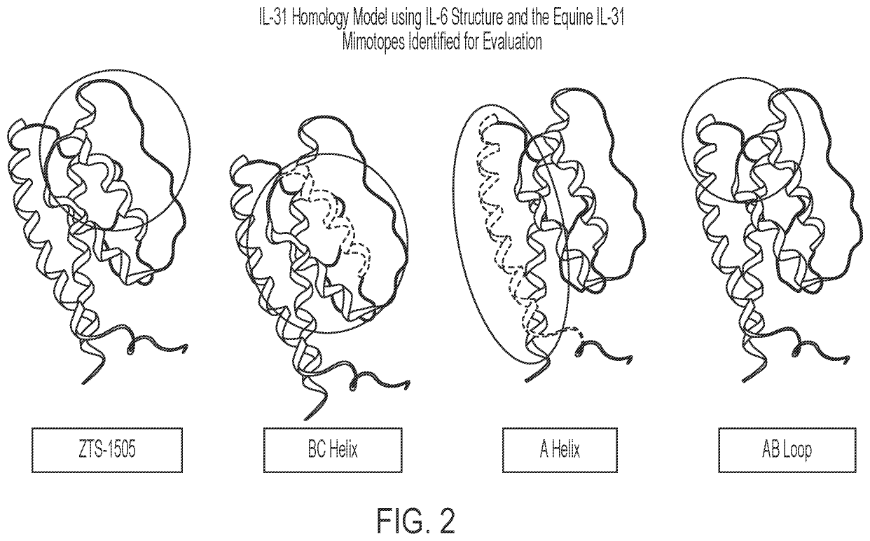

IL-31 mimotopes were designed and generated based on known neutralizing epitopes identified during dog and cat studies. Several IL-31 mimotope constructs, constrained as well as linear, were evaluated, including the ZTS-7240 mimotope (constrained with linker) corresponding to the 1505 region (SEQ ID NO:3, SEQ ID NO:4, and SEQ ID NO:5), the ZTS-7241 mimotope corresponding to the A helix (SEQ ID NO:6), the ZTS-765 mimotope corresponding to the BC helix (SEQ ID NO:2) and the ZTS-7242 mimotope corresponding to the AB loop (SEQ ID NO:7) of the IL-31 homology model (FIG. 2). The mimotopes which were evaluated for their ability to reduce the pruritus induced by the equine IL-31 polypeptide are shown in Table 3 of the Example section. The sequence identifier numbers in Table 3 correspond to the IL-31 mimotope amino acid sequences but exclude the C-terminus Cysteines used for chemical conjugation purposes, linkers, and acetyl groups depicted in Table 3 which were also a part of the evaluated mimotopes.

The 15H05 epitope binding region was previously disclosed in US2019/0284272 A1 (Zoetis Services LLC). The 15H05 may be alternatively referred to herein as 1505 at least because they share the same CDRs. The 15H05/1505 epitope region is selected from at least one of the following: a) a region between about amino acid residues 124 and 135 of a feline IL-31 sequence represented by SEQ ID NO: 8 (Feline_IL31_wildtype); b) a region between about amino acid residues 124 and 135 of a canine IL-31 sequence represented by SEQ ID NO: 9 (Canine_IL31); and c) a region between about amino acid residues 118 and 129 of an equine IL-31 sequence represented by SEQ ID NO: 10 (Equine_IL31).

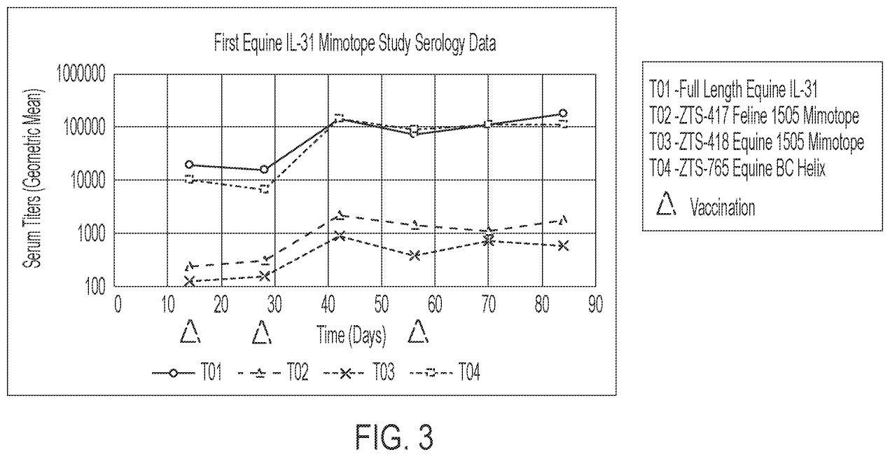

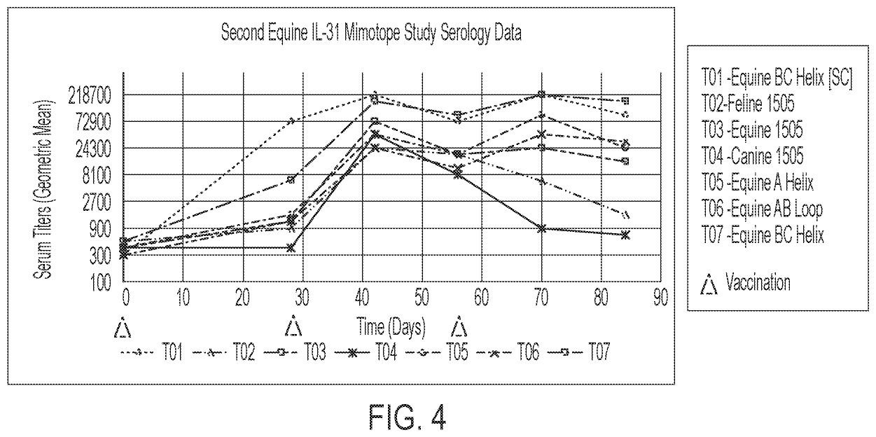

In vivo horse serology studies (Table 4, FIG. 3, Table 5, FIG. 4) were conducted to evaluate carrier polypeptide-conjugated IL-31 mimotopes and to compare with serological titers elicited by full length equine IL-31. In these studies, the carrier polypeptide was CRM 197. However, the present invention is not limited to this carrier polypeptide. Squalane-Pluronic (SP) oil+CpG oligodeoxynucleotide (CpG) was used as the adjuvant for these vaccinations. However, the present invention is not limited to this particular adjuvant mixture. Serology data from these studies (FIGS. 3 and 4) demonstrated that vaccine compositions comprised of these equine IL-31 mimotope constructs can elicit strong antibody titers and that the titers were comparable to that elicited by the full-length equine IL-31.

In follow up in vivo studies, these vaccinated horses were challenged with equine IL-31 to determine if resulting antibodies were capable of preventing or reducing pruritis. Based on the high antibody titers elicited by horses vaccinated with CRM197 conjugated IL 31 mimotopes (FIG. 4), horses from the serology studies were utilized in a challenge study using the IL-31 itch model developed. Selected horses were vaccinated with an additional boost of the corresponding mimotope vaccines, after conclusion of the serology study, and were challenged with IL-31 at Day 14 after the final boost and at Day 105 after the final boost.

Among the candidate equine IL-31 mimotope constructs which were evaluated were ZTS-7240 (includes SEQ ID NO:4) mimotope (constrained) corresponding to the 1505 region and ZTS-7241 (includes SEQ ID NO:6) mimotope (linear) corresponding to the A helix of the IL-31 homology model (FIG. 2). IL-31 mimotope vaccine compositions including the ZTS-7240 and ZTS-7241 mimotopes, demonstrated 80% and 68% reduction in itch (relative to historic scores), respectively, in vaccinated horses, after 105 days of the final booster dose (Table 7; Table 8).

Since the IL-31 mimotope vaccines were designed based on known neutralizing monoclonal antibody epitopes (e.g., monoclonal antibody (mAb) 1505 disclosed in US2019/0284272 A1 for which the applicant is Zoetis Services LLC), it is highly likely that such neutralizing antibodies targeting the horse IL-31 1505 mAb epitope would also provide protection to IL-31 induced pruritis in the horse model. Also, evidence can be provided from the anti-pruritic activity of caninized 34D03 mAb which was evaluated using a canine model of IL-31-induced pruritus (U.S. Pat. No. 10,526,405 B2 to Mann et al.). With the dog model, a 1.5 μg/kg intravenous challenge dose of recombinant canine IL-31 known to induce a transient period of pruritic behavior in beagle dogs (IL-31 challenge, pruritus duration <24 hour) was repeatedly delivered to animals before and up to 63 days after a single 1.0 mg/kg SC dose of CAN 34D03.

Pruritic scores were generated at each time period under evaluation by making “yes/no” determinations as to whether a pruritic behavior was displayed over consecutive 1 minute time-intervals (maximal pruritic score=30 for each baseline period; 120 for the post-IL-31 challenge period). Pruritic scores were obtained before and after CAN 34D03 treatment, which was given on day 0 of the study. Seven days prior to mAb treatment, the mean post-IL-31 challenge pruritic score of the dogs was 68±13 (S.E., n=4). By comparison, on study days 7, 14, 21, the mean post-IL-31 challenge pruritic scores had lowered to 5±2, 8±4, and 9±5, respectively. These changes in pruritic score between day −7 and days 7-21 represent a ≥85%, decrease in overall pruritic reactivity to IL-3. These reduced pruritic scores are similar to the reduction induced by the horse IL-31 mimotope vaccines, therefore it can be strongly assumed that evaluation of equinized monoclonal antibodies, raised against neutralizing IL-31 epitopes, would provide similar reduction in pruritis scores when administered to the horse IL-31 itch model.

To evaluate the effect of oclacitinib maleate in the developed equine model disclosed herein and to further demonstrate model utility, horses challenged with equine IL-31 were administered oclacitinib maleate and the effect on reduction on pruritis was determined. Oclacitnib maleate is a known janus kinase inhibitor disclosed in U.S. Pat. No. 8,133,899 to Mitton-Fry et al. The intravenous solution of recombinant equine IL-31 was prepared as described in Example 4. Oclacitinib maleate was combined with a feed carrier in Example 4. Horses were administered either feed carrier only or oclacitnib maleate at a determined number of hours prior to equine IL-31 challenge administration. Post-challenge, pruritic activity of the horses was observed for 120 minutes, although the model is not limited to this observation period.

In order to characterize pruritic behavior in the study described in Example 4, horses were observed for any behavior that can be identified as pruritus. Again, signs of pruritus in horses include the following: biting or scratching of self, rubbing against objects, feet stomping, tail flicking, head or body shaking, rolling, twitching of skin, and combinations thereof.

A “yes” response was indicated by marking a “1” in the space provided for the specific behavior during the one minute interval. The cumulative number of yes responses per behavior were added to determine a cumulative pruritus score for each horse. Following IL-31 challenge, horses were observed for 120 minutes to determine a post challenge pruritus score.

Horses challenged with IL-31 following 0.25 mg/kg oclacitinib maleate treatment showed a reduction in pruritic activity when compared to the placebo horses (Table 10). Horses challenged 1.5 hours after oclacitinib maleate treatment (T02) had a reduction in average pruritic score by 30% when compared to the placebo group (T01). Horses challenged 21.5 hours after oclacitinib maleate treatment (T03) had a reduction in average pruritic score by 29% when compared to the placebo group (T01). Horses challenged 4.5 hours after oclacitinib maleate treatment (T04) had a reduction in average pruritic score by 31% when compared to the placebo group (T01).

The invention will now be described further by the non-limiting examples below.

### EXAMPLES

### Example 1

### Establishment and Characterization of an Equine IL-31 Induced Pruritus Model Via IL-31 Challenge

Prior to the present invention, it was not known if IL-31 plays a key role in equine pruritus, a key clinical sign of allergic skin disease in horses. Thus, an IL-31 induced pruritus model was established in horses, as a surrogate for naturally occurring clinical disease. In this study, five concentrations of equine IL-31 were used to induce pruritus in horses. These doses were 1 μg/kg, 0.5 μg/kg, 0.25 μg/kg, 0.1 μg/kg and 0.05 μg/kg. The goal of this model is to induce a strong pruritic response without having too high of concentrations that a therapeutic cannot overcome the pruritic behavior or too strong of a pruritic response that the animals are uncomfortable.

Equine DNA IL-31 sequence was designed, optimized and synthesized. The complete sequence was sub-cloned into pcDNA3.4vector. Transfection grade plasmid was maxi-prepared for Expi293F cell expression. The designed, optimized, and synthesized DNA sequence encoding the recombinant equine IL-31 protein comprised the following nucleotide sequence:

(SEQ ID NO: 1)
ATGGGCTGGTCCTGCATCATTCTGTTTCTGGTGGCCACAGCCACCGGCGT
GCACTCTGGACCTATCTATCAGCTGCAGCCCAAAGAGATCCAGGCCATCA
TCGTGGAACTGCAGAACCTGAGCAAGAAGCTGCTGGACGACTACCTGAAC
AAAGAAAAGGGCGTGCAGAAGTTCGACAGCGACCTGCCTAGCTGCTTCAC
CAGCGATTCTCAGGCCCCTGGCAACATCAACAGCAGCGCCATCCTGCCTT
ACTTCAAGGCCATCTCTCCCAGCCTGAACAACGACAAGAGCCTGTACATC
ATCGAGCAGCTGGACAAGCTGAACTTCCAGAACGCCCCTGAAACCGAGGT
GTCCATGCCTACCGACAACTTCGAGCGGAAGCGGTTCATCCTGACCATCC
TGCGGTGGTTCAGCAACTGCCTGGAACACAGAGCCCAGCACCACCACCAT
CACCATTGATAAGCTT.

The recombinant equine IL-31 polypeptide encoded by the nucleotide sequence of SEQ ID NO: 1 is as follows:

(SEQ ID NO: 10)
MGWSCIILFLVATATGVHSGPIYQLQPKEIQAIIVELQNLSKKLLDDYLN
KEKGVQKFDSDLPSCFTSDSQAPGNINSSAILPYFKAISPSLNNDKSLYI
IEQLDKLNFQNAPETEVSMPTDNFERKRFILTILRWFSNCLEHRAQHHHH
HH.

However, it is understood that the signal sequence is usually removed in the mature protein. The predicted signal sequence is underlined above in SEQ ID NO: 10. The recombinant equine IL-31 protein was expressed transiently in suspension EXPICHO-S cells which were maintained in EXPICHO expression medium (Gibco) between 0.14 and 8.0×10e6 cells/ml. Cells are diluted following the ExpiCHO Protocol user manual on Day −1 and transfection day. Diluted cells are transfected as described in the protocol using reagents sourced from ExpiFectamine CHO Transfection Kit (Gibco) following Max Titer conditions. The 2 L bulk culture volume was aliquoted and transfected in 8×1 L Corning flasks, each containing 200 ml culture. Following 12-14 days of incubation, the cultures were harvested, pooled and clarified and about 2.2 L of supernate was delivered for purification.

The filtered supernate was adjusted to ˜500 mM NaCl, 5 mM imidazole, and pH 7.4, before batch loading onto 25 mL of Ni Sepharose Excel resin, pre-equilibrated with 5 mM imidazole, 20 mM sodium phosphate, 500 mM NaCl, pH 7.4. Sample and resin were allowed to mix (with a suspended stir bar) at 4° C. overnight. The unbound fraction was then filtered off, the resin packed in an XK26 column, and hooked up to an AKTA Pure chromatography system. The histidine tagged protein was eluted via linear gradient from 5 to 500 mM imidazole, each in the same buffer. A pool of fraction was formed based on SDS-PAGE, dialyzed against 20 mM CH3COONa, 150 mM NaCl, pH 5.0. The resulting sample was found to be ˜86% pure on SDS-PAGE. Results of mass spectrometry were consistent with masses expected for the theoretical sequence. Final yield was 284 mg/L. The protein was aliquoted, snap frozen, and stored at −80 C until further use.

### Equine IL-31 Material

Concentration: 5 mg/mL
Formulation: 20 mM CH3COONa, 150 nM NaCl, pH 5.0
Storage: −80° C.

### IL-31 Challenge Material

Vehicle: phosphate buffered saline
Dose: 0.05 μg/kg-1.0 μg/kg
IL-31 concentration: 0.1 mg/mL-0.62 mg/mL

### Animals:

Species/strain/breed: Horse
Sex: Intact females/Castrated males
Initial age: >2 years
Initial Weight: 400-700 kg
Origin: Tri-Pine Stockfarm
Number (n): 15
Identification: Each horse is uniquely identified by collar number
Feeding: Grain limit fed as appropriate. Hay/hay cubes and pasture provided ad libitum
Watering: Water provided ad libitum
Housing: Group housed in a paddock. Horses are single housed in stalls during observation periods.

### Experimental Design

Three to eight horses were tested at one time. Dose level depended on the pruritic response from the previous dosing group. The final desired dose was repeated. The final study design was as follows (Table 1):

TABLE 1
Equine Induced Pruritus Model Design via IL-31 Challenge

| | Dose Level | | | |
|---|---|---|---|---|
| Treatment | (ug/kg) | N | Treatment | Route |
| T01 | 1 | 3 | IL-31 | IV |
| T02 | 0.5 | 7* | IL-31 | IV |
| T03 | 0.25 | 15* | IL-31 | IV |
| T04 | 0.1 | 8 | IL-31 | IV |
| T05 | 0.05 | 8 | IL-31 | IV |

*Testing was completed over 2 days

### Randomization

No randomization was required for this study.

### Procedure/Methods

### Inclusion/Exclusion Criteria

Animals were in overall good health and deemed suitable for the study based on a physical examination performed by a clinical veterinarian prior to administration of test article.

Horses included on study did not received a non-steroidal anti-inflammatory drug within 7 days, short acting corticosteroid within 14 days, intermediate/long-acting or repository corticosteroids within 30 days or any other drug within five days prior to the pre-study physical examination.

### Body Weight

Body weight was collected for dose determination of IL-31 challenge. Body weight information was collected within 14 days prior to administration of IL-31 challenge.

### Day −7 Acclimation Period

Horses were moved to the designated pasture for minimum acclimation period of 7 days prior to Day 0. During this period, horses were acclimated to the individual stalls that will be used for challenge administration and pruritus observation at least once per day as needed.

### Baseline IL-31 Blood Collection

Blood samples for measurement of serum IL-31 concentrations were collected at least 3 days prior to the IL-31 challenge.

### Evaluation for any Dermatological Lesions

Horses were evaluated for active dermatologic lesions on the day of IL-31 challenge. Active lesions can include, but are not limited to, open wounds, wheals, urticaria, papules, and ulcerations. Any lesions were documented. Minor bumps and scratches were documented but no horse was excluded due to lesions.

### Acclimation to Observation Stall

On the scheduled IL-31 challenge days, animals were led to observation stalls. The horses were single-housed in separate stalls. Horses were acclimated to the observation stall for at least one hour prior to administration of IL-31 challenge. Horses were fed hay cubes in the observation stall.

### Baseline Pruritus Score

Observations of normal pruritic behavior (baseline pruritus scores) were made prior to the administration of IL-31 challenge. The horses were scored the same was as post challenge observations except no IL-31 challenge was administered and observation of normal pruritic behavior was made for 30 minutes.

### Administration of IL-31 Challenge

Following baseline pruritus score, horses were administered the IL-31 challenge. IL-31 challenge was administered intravenously to each animal via a jugular vein. The horse ID and time of administration was recorded. Recording of pruritic activity began approximately 15 to 25 minutes after the last of the horses is administered the IL-31 challenge (Table 2 (A to F) below and FIG. 1).

### Characterization of Pruritic Behavior

In order to characterize pruritic behavior, horses were observed for any behavior that can be identified as pruritus. Classical signs of pruritus in horses include:
Biting or scratching at self
Rubbing against objects
Feet stomping
Tail flicking
Head or body shaking
Rolling
Twitching of skin

A “yes” response was indicated by marking a “1” and a “no” response was indicated by marking a “0” during the one minute interval. The cumulative number of yes responses were added to determine a cumulative Pruritus Score for each horse.

### Post Challenge Observation Period

Following each IL-31 challenge, horses were observed for a 120-minute period to determine a post challenge pruritus score.

### Animal Health Observations

No adverse events of anaphylaxis were noted. The first three horses dosed at 1.0 μg/kg were given diphenhydramine following the post challenge observation period since the horses experienced an uncomfortable level of pruritus and needed treatment.

### Results

TABLE 2
Pruritus Scores for Horses Pre and Post IL-31 Challenge at IL-31
Concentrations Ranging from 0.05 μg/kg to 1.0 μg/kg
A. Dose 1.0 μg/kg

| | Horse ID | 640 | 639 | 45 | Average |
|---|---|---|---|---|---|
| Pre- | Pruritus | 7 | 11 | 7 | 8.3 |
| Challenge | Score/30 | | | | |
| | Pruritus % | 23% | 37% | 23% | 28% |
| Post | Pruritus | 120 | 120 | 104 | 114.7 |
| Challenge | Score/120 | | | | |
| | Pruritus % | 100% | 100% | 87% | 96% |

B. Dose 0.5 μg/kg

| | Horse ID | 45 | 51 | 54 | 55 | 638 | 639 | 640 | Average |
|---|---|---|---|---|---|---|---|---|---|
| Pre- | Pruritus | 19 | 2 | 16 | 11 | 5 | 13 | 1 | 9.6 |
| Challenge | Score/30 | | | | | | | | |
| | Pruritus % | 63% | 7% | 53% | 37% | 17% | 43% | 3% | 32% |
| Post | Pruritus | 120 | 70 | 117 | 106 | 93 | 120 | 110 | 105.1 |
| Challenge | Score/120 | | | | | | | | |
| | Pruritus % | 100% | 58% | 98% | 88% | 78% | 100% | 92% | 88% |

C. Dose 0.25 μg/kg

| | Horse ID | 45 | 51 | 54 | 55 | 638 | 639 | 640 | Average |
|---|---|---|---|---|---|---|---|---|---|
| Pre- | Pruritus | 13 | 3 | 26 | 5 | 4 | 9 | 7 | 9.6 |
| Challenge | Score/30 | | | | | | | | |
| | Pruritus % | 43% | 10% | 87% | 17% | 13% | 30% | 23% | 32% |
| Post | Pruritus | 95 | 84 | 109 | 81 | 86 | 113 | 113 | 97.3 |
| Challenge | Score/120 | | | | | | | | |
| | Pruritus % | 79% | 70% | 91% | 68% | 72% | 94% | 94% | 81% |

D. Dose 0.1 μg/kg

| | Horse ID | 45 | 51 | 54 | 55 | 638 | 639 | 640 | 608 | Average |
|---|---|---|---|---|---|---|---|---|---|---|
| Pre- | Pruritus | 16 | 5 | 14 | 29 | 6 | 5 | 16 | 7 | 12.3 |
| Challenge | Score/30 | | | | | | | | | |
| | Pruritus % | 53% | 17% | 47% | 97% | 20% | 17% | 53% | 23% | 41% |
| Post | Pruritus | 62 | 72 | 115 | 74 | 71 | 86 | 96 | 103 | 84.9 |
| Challenge | Score/120 | | | | | | | | | |
| | Pruritus % | 52% | 60% | 96% | 62% | 59% | 72% | 80% | 86% | 71% |

E. Dose 0.05 μg/kg

| | Horse ID | 571 | 577 | 579 | 583 | 590 | 595 | 597 | 599 | Average |
|---|---|---|---|---|---|---|---|---|---|---|
| Pre- | Pruritus | 3 | 1 | 17 | 2 | 27 | 6 | 2 | 8 | 8.3 |
| Challenge | Score/30 | | | | | | | | | |
| | Pruritus % | 10% | 3% | 57% | 7% | 90% | 20% | 7% | 27% | 28% |
| Post | Pruritus | 50 | 36 | 67 | 43 | 25 | 42 | 45 | 37 | 43.1 |
| Challenge | Score/120 | | | | | | | | | |
| | Pruritus % | 42% | 30% | 56% | 36% | 21% | 35% | 38% | 31% | 36% |

F. Dose 0.25 μg/kg

| | Horse ID | 573 | 575 | 581 | 585 | 589 | 591 | 593 | 600 | Average |
|---|---|---|---|---|---|---|---|---|---|---|
| Pre- | Pruritus | 1 | 0 | 2 | 1 | 0 | 4 | 7 | 18 | 4.1 |
| Challenge | Score/30 | | | | | | | | | |
| | Pruritus % | 3% | 0% | 7% | 3% | 0% | 13% | 23% | 60% | 14% |
| Post | Pruritus | 56 | 20 | 11 | 87 | 68 | 86 | 52 | 81 | 57.6 |
| Challenge | Score/120 | | | | | | | | | |
| | Pruritus % | 47% | 17% | 9% | 73% | 57% | 72% | 43% | 68% | 48% |

### Discussion of Example 1 Results

The cytokine IL-31 has been implicated in pruritus and has therefore been used to build models of pruritus associated with allergic dermatitis, including in mice and dogs. This cytokine is secreted by CD4⁺ T cells, and when bound to its receptor, activates a number of pathways including those in peripheral nerves to induce pruritic behavior. The equine model described herein will serve as a proof of concept model that can be used to determine dose and efficacy for a potential anti-pruritic drug for use in equine mammals (e.g. small molecule, monoclonal antibody or IL-31 mimetic vaccine)

The objective of this study was to determine a dose of IL-31 that would consistently elicit the appropriate pruritic response to allow for therapeutic testing in the future. Of the five doses of IL-31 tested in this study, it was determined that 0.25 μg/kg was a preferred dose. On average, 0.25 μg/kg increased pruritus from 32% at baseline to 81% post challenge. The 1 μg/kg dose was not a preferred option due to the need for diphenhydramine to make the horses more comfortable after the post challenge observations. In the 0.5 μg/kg group, there were still some horses that had post challenge pruritus scores of 100% therefore this dose was not optimal. The 0.1 μg/kg dose had a reasonable pruritic response with an average baseline pruritic score of 41% and an average post challenge pruritic score of 71% but it was not as robust of a response as the 0.25 μg/kg dose. The 0.05 μg/kg did not elicit a strong pruritic response with only an average post challenge pruritic score of 48%.

### Example 2

### Equine IL-31 Serology Studies after Administration of IL-31 Mimotope Vaccines

These studies were conducted to screen and evaluate the ability of various CRM197 conjugated equine IL-31 mimotope vaccines to elicit a serological response in horses.

The equine IL-31 mimotopes were designed and generated based on neutralizing epitopes which had been identified during dog and cat studies. Canine, feline, equine, as well as human IL-31 vaccine mimotopes were disclosed in US 2019/0282704 A1 (Zoetis Services LLC).

The IL-31 mimotopes used in the present studies are shown in Table 3 below.

TABLE 3
IL-31 mimotopes used in the present studies

| Peptide | | |
|---|---|---|
| Code | Description | Peptide Sequence (N to C terminus) |
| N/A | Full length equine IL- | SEQ ID NO: 10 less signal sequence |
| | 31 | |
| ZTS-417 | Feline 1505 mimotope | Ac-CAKVSMPADNFERKNFILTC |
| | | (SEQ ID NO: 3) |
| ZTS-418 | Equine 1505 | Ac-CTEVSMPTDNFERKRFILTC |
| | mimotope | (SEQ ID NO: 4) |
| ZTS-765 | Equine BC helix | Ac-CGSGNSSAILPYFKAISPSLNNDKSLYIIEQLDKLNF-NH2 |
| | mimotope | (SEQ ID NO: 2) |
| ZTS-422 | Feline 1505 mimotope | Ac-C(Ahx]C(mT2b)AKVSMPADNFERKNFILTC(mT2b)-NH2 |
| | | (SEQ ID NO: 3 with mT2b linker) |
| ZTS-7240 | Equine1 505 | Ac-C[Ahx]C(mT2b)TEVSMPTDNFERKRFILTC(mT2b)-NH2 |
| | mimotope | (SEQ ID NO: 4 with mT2b linker)) |
| ZTS-564 | Canine 1505 | Ac-C(mT2b)TEISVPADTFECKSFILTC(mT2b)-NH2 |
| | mimotope | (SEQ ID NO: 5 with mT2b linker) |
| ZTS-7241 | Equine Helix A | Ac-CGSGGPIYQLQPKEIQAIIVELQNLSKK-NH2 |
| | mimotope | (SEQ ID NO: 6) |
| ZTS-7242 | Equine AB loop | Ac-CGSGKEKGVQKFDS-NH2 |
| | mimotope | (SEQ ID NO: 7) |

*Terminal Cysteines (C) annotated in bold and underlined were added to facilitate conjugation chemistry using the free thiol groups. Also, ZTS-765, ZTS-7241, and ZTS-7242 contain a three-amino acid spacer (GSG) annotated with double underlined text. “Ahx” stands for aminohexanoic acid and “Ac” stands for acetyl group. The mT2b linker was previously described in US 2019/0282704 A1.

Several equine IL-31 mimotope constructs, constrained as well as linear, were evaluated, including the ZTS-7240 (comprising SEQ ID NO:4) mimotope (constrained with mT2b linker) corresponding to the 1505 region, ZTS-7241 (comprising SEQ ID NO:6) mimotope (linear) corresponding to the A helix, the ZTS-765 (comprising SEQ ID NO:2) mimotope (linear) corresponding to the BC helix and the ZTS-7242 (comprising SEQ ID NO:7) mimotope (linear) corresponding to the AB loop of the IL-31 homology model (FIG. 2). In addition, a few other mimotopes corresponding to the 1505 region of feline and canine IL-31 were also evaluated in these studies (ZTS-422 comprising SEQ ID NO:3 and ZTS-564 comprising SEQ ID NO:5, respectively). The mT2b linker was previously described in US 2019/0282704 A1.

An equine study (Table 4 below, FIG. 3) was conducted to evaluate CRM197 conjugated IL-31 mimotopes (linear and generic linker) and to compare with serological titers elicited by full length equine IL-31. Squalane-Pluronic (SP) oil+CpG oligodeoxynucleotide (CpG) was used as the adjuvant for each of these vaccinations. Also, the dose volume (mL) for each of these vaccinations was 1 mL.

TABLE 4
Study Design for First Equine Serology Study After Administration of
IL31 Mimotope Vaccines

| | | | | | Dose |
|---|---|---|---|---|---|
| Treatment | Mimotope | | | | Vol- |
| Groups | with CRM197 | Mimotope | | | ume |
| (n = 4) | (25 μg/dose) | Description | Adjuvant | Route | (mL) |
| T01 | N/A | Full length equine | SP oil + | SC | 1 |
| | | IL-31 | CpG | | |
| T02 | ZTS-417 | Feline 1505 | SP oil + | SC | 1 |
| | | mimotope | CpG | | |
| T03 | ZTS-418 | Equine 1505 | SP oil + | SC | 1 |
| | | mimotope with | CpG | | |
| T04 | ZTS-765 | Equine BC helix | SP oil + | SC | 1 |
| | | mimotope | CpG | | |

A second equine serology study (Table 5 below, FIG. 4) was conducted to evaluate additional CRM197 conjugated IL-31 mimotopes (constrained and non-constrained). Squalane-Pluronic (SP) oil+CpG oligodeoxynucleotide (CpG) was used as the adjuvant for each of these vaccinations. Also, the dose volume (mL) for each of these vaccinations was 1 mL.

TABLE 5
Study Design for Second Equine Serology Study After Administration of
IL-31 Mimotope Vaccines

| | | | | | Dose |
|---|---|---|---|---|---|
| Treatment | Peptide with | | | | Vol- |
| Group | CRM197 | Mimotope | | | ume |
| (n = 4) | (25 ug/dose) | Description | Adjuvant | Route | (mL) |
| T01 | ZTS-765 | Equine BC Helix | SP oil + CpG | SC | 1 |
| | | mimotope | | | |
| T02 | ZTS-422 | Feline 1505 | SP oil + CpG | IM | 1 |
| | | mimotope | | | |
| T03 | ZTS-7240 | Equine 1505 | SP oil + CpG | IM | 1 |
| | | mimotope | | | |
| T04 | ZTS-564 | Canine 1505 | SP oil + CpG | IM | 1 |
| | | mimotope | | | |
| T05 | ZTS-7241 | Equine A Helix | SP oil + CpG | IM | 1 |
| | | mimotope | | | |
| T06 | ZTS-7242 | Equine AB Loop | SP oil + CpG | IM | 1 |
| | | mimotope | | | |
| T07 | ZTS-765 | Equine BC Helix | SP oil + CpG | IM | 1 |
| | | mimotope | | | |

Vaccination: 3 doses on days 0, 28, & 56

### Discussion of Example 2 Results

Serology data from these studies (FIGS. 3 and 4) demonstrated that equine IL-31 mimotope constructs can elicit strong antibody titers and that the titers were comparable to that elicited by the full-length equine IL-31.

### Example 3

### Equine Challenge Study Using the IL-31 Itch Model after Administration of IL-31 Mimotope Vaccines

Based on the high antibody titers elicited by horses vaccinated with CRM197 conjugated IL 31 mimotopes (FIG. 4), horses from the serology studies were utilized in a challenge study using the IL-31 itch model developed by the present inventors. Selected horses were vaccinated with an additional boost of the corresponding mimotope vaccines, after conclusion of the serology study, and were challenged with IL-31 at Day 14 after the final boost and at Day 105 after the final boost.

Various candidate IL-31 mimotope constructs were evaluated, including ZTS-7240 (comprising SEQ ID NO:4) mimotope (constrained) corresponding to the 1505 region and ZTS-241 (comprising SEQ ID NO:6) mimotope (linear) corresponding to the A helix of the IL-31 homology model (FIG. 2). Study design is outlined in Table 6 below:

TABLE 6
Study Design for IL-31 Challenge Study After Vaccination
with IL-31 Mimotope Vaccines

| | | Peptide | | | | |
|---|---|---|---|---|---|---|
| | | with | | | | |
| Treat- | | CRM197 | | | | Dose |
| ment | Animal | (25 μg/ | Mimotope | | | Volume |
| Group | IDs | dose) | Description | Adjuvant | Route | (mL) |
| T01 | 595 | ZTS-765 | Equine BC | SP oil + | SC | 1 |
| | 600 | | Helix | CpG | | |
| | | | mimotope | | | |
| T03 | 45 | ZTS-7240 | Equine 1505 | SP oil + | IM | 1 |
| | 619 | | mimotope | CpG | | |
| | 639 | | | | | |
| T05 | 54 | ZTS-7241 | Equine A | SP oil + | IM | 1 |
| | 608 | | Helix | CpG | | |
| | 630 | | mimotope | | | |
| T06 | 55 | ZTS-7242 | Equine AB | SP oil + | IM | 1 |
| | 597 | | Loop | CpG | | |
| | 638 | | mimotope | | | |
| T07 | 573 | ZTS-765 | Equine BC | SP oil + | IM | 1 |
| | 589 | | Helix | CpG | | |
| | 598 | | mimotope | | | |
| T08 | 583 | None | None | None | None | None |
| | 592 | | | | | |

Vaccination: Single booster dose given on Day 0
Challenge: 1st challenge: Day 14 post-boost; 2nd Challenge: Day 105 post-boost

There were no treatment groups T02 and T04 assigned in this study in order to continue to keep the group designations of respective horses from the first serology study. One horse each from treatment groups T02 and T04 from the first serology were used in treatment group T08 as negative controls (No booster dose given; challenged on Day 14 and Day 105 along with other treatment groups).

ZTS-7240 (comprising SEQ ID NO:4) and ZTS-7241 (comprising SEQ ID NO:6) demonstrated 80% and 68% reduction in itch (relative to historic scores), respectively in vaccinated horses, after 105 days of the final booster dose (Table 7; Table 8).

TABLE 7
IL-31 Itch Model: Pruritus Scores (Individual Horse Data)

| | | Histor- | 1st | | 2nd | |
|---|---|---|---|---|---|---|
| Treat- | Animal | ical | Challenge | Percent | Challenge | Percent |
| ment | ID | Score* | Score | Change | Score | Change |
| T01 | 595 | 67.5 | 17 | −74.81% | 41 | −39.26% |
| T01 | 600 | 104 | 11 | −89.42% | 91 | −12.50% |
| T03 | 619 | 105.5 | 18 | −82.94% | 24 | −77.25% |
| T03 | 639 | 102.3 | 6 | −94.13% | 8 | −92.18% |
| T03 | 45 | 90.3 | 21 | −76.74% | 15 | −83.39% |
| T05 | 630 | 88.5 | 12 | −86.44% | 11 | −87.57% |
| T05 | 608 | 97.5 | 5 | −94.87% | 50 | −48.72% |
| T05 | 54 | 109.3 | 38 | −65.23% | 33 | −69.81% |
| T06 | 638 | 69.7 | 53 | −23.96% | — | Not |
| | | | | | | Done |
| T06 | 597 | 54.5 | 45 | −17.43% | 67 | 22.94% |
| T06 | 55 | 67.8 | 54 | −20.35% | 115 | 69.62% |
| T07 | 589 | 73.3 | 4 | −94.54% | 56 | −23.60% |
| T07 | 598 | 98 | 33 | −66.33% | 58 | −40.82% |
| T07 | 573 | 75 | 21 | −72.00% | 22 | −70.67% |
| T08 | 583 | 94 | 120 | 27.66% | 116 | 23.40% |
| T08 | 592 | 110.3 | 120 | 8.79% | 113 | 2.45% |

*Historical Score was established in these horses about 15 months before the first challenge.

TABLE 8
IL-31 Itch Model: Pruritus Scores (% Mean Change in Scores)

| | 1st Challenge | 2nd Challenge |
|---|---|---|
| Treatment | Average % Change | Average % Change |
| T01 | −82.12% | −25.88% |
| T03 | −84.61% | −84.27% |
| T05 | −82.18% | −68.70% |
| T06 | −20.58% | 46.28% |
| T07 | −77.62% | −45.03% |
| T08 | 18.23% | 12.93% |

### Example 4

### Pharmacokinetics and Pharmacodynamics of Oclacitinib Maleate in Equine Model of IL-31 Induced Pruritus

In the present example, a small molecule compound was assessed for its ability to reduce pruritus in the equine model of horse IL-31 induced pruritus described herein. The specific test substance was Oclacitinib maleate which was administered to the horses in a feed carrier.

Test Substances

| | |
|---|---|
| Test Substance: | Oclacitinib maleate |
| Source: | Zoetis |
| Potency: | 16 mg tablets* |
| Storage conditions: | Stored per label directions: Stored at controlled room |
| | temperature 15° C. to 30° C. (59^{o} F. to 86° F.) |

*16 mg tablets are 16 mg of Oclacitinib as Oclacitinib maleate

Placebo

| | |
|---|---|
| Control Substance: | Carrier substance only |

Carrier

| | |
|---|---|
| Carrier Substance: | Sweet Feed Omelene 300 |
| Source: | Purina |
| Potency: | 0 mg |
| Lot number: | NA |
| Storage conditions: | Stored in a dry well ventilated area protected from |
| | rodents and insects. |

Challenge Material

| | |
|---|---|
| Challenge Material: | Equine IL-31 (mature version of polypeptide |
| | encoded by SEQ ID NO: 1) |
| Source: | Zoetis |
| Potency: | 5 mg |
| Storage conditions: | Store at −80° C. |

### IL-31 Challenge Preparation

The intravenous solution was prepared from stock concentrations of equine recombinant IL-31. An aliquot of IL-31 was thawed immediately prior to use and diluted with vehicle (phosphate buffered saline without Ca++ or Mg++) such that a standard dose of the cytokine was delivered to each horse in a total volume of mL.

### Formulations

Oclacitinib maleate was combined with carrier at the time of dosing, and mixed thoroughly to a total weight as stated below. Only whole tablets were used, doses were rounded to the nearest 16 mg.

| Name | Carrier | Total Volume |
|---|---|---|
| Placebo (carrier only) | Sweet Feed Omelene 300 | 1-2 L |
| Oclacitinib maleate tablets | Sweet Feed Omelene 300 | 1-2 L |

Animals

| | |
|---|---|
| Species, Breed and/or Strain: | Horse |
| Sex and Number of Animals: | Intact females/castrated males |
| Age Range at Dose Initiation: | >2 years |
| Weight Range at Dose | ~450-650 kg |
| Initiation: | |
| Method of Animal Identification: | Each horse is uniquely identified |
| | by collar number |
| Health Evaluation: | No apparent health abnormalities |
| | prior to initiation of dosing |
| Housing: | Group housed in a paddock. Horses |
| | were single housed in stalls during |
| | the observation periods |
| Acclimation: | Horses were acclimated to stalls at |
| | least 5 times prior to study. On |
| | challenge days, horses were |
| | acclimated to stalls for at least 1 |
| | hour prior to baseline observations. |
| Diet: | Grain limit fed as appropriate. Hay/ |
| | hay cubes and pasture provided ad |
| | Water provided ad libitum. |

### Study Design

Horses were administered either carrier only or oclacitinib maleate tablets at a determined number of hours prior to IL-31 challenge administration. Post-challenge, pruritic activity of the horses was observed for 120 minutes. Blood samples for pharmacokinetic analysis were collected at the time of IL-31 administration and immediately following the post-challenge observation period. Due to limited number of observation stalls, horses were challenged in batches. Batch 1 will include T01 (n=2)+T02 (n=5). Batch 2 will include T01 (n=2)+T03 (n=5). Batch 3 will include T01 (n=2)+T04 (n=5) (Table 9)

TABLE 9
Study Design for Equine IL-31 Itch Model Used to Investigate the
Pharmacokinetics and Pharmacodynamics of Oclacitinib Maleate

| | | | | IL-31 | Challenge | Post Test |
|---|---|---|---|---|---|---|
| | | | | Chal- | Time | Article |
| | | | | lenge | Post Test | Dose |
| | | Test | Dose | Dose | Article | Observation |
| Group | N | Article | (mg/kg) | (mcg/kg) | Dosing | Period |
| T01 | 6* | Placebo | 0 | 0.25 | 1.5 hr, | 2-4 hr, |
| | | | | | 21.5 hr | 22-24 hr |
| T02 | 5 | Oclacitinib | 0.25 | 0.25 | 1.5 hr | 2-4 hr |
| | maleate | | | | | |
| T03 | 5 | Oclacitinib | 0.25 | 0.25 | 21.5 hr | 22-24 hr |
| | maleate | | | | | |
| T04 | 5 | Oclacitinib | 0.25 | 0.25 | 4.5 hr | 5-7 hr |
| | maleate | | | | | |

*2 Placebo (T01) horses were tested with each treatment group (T02, T03, T04).

### Randomization

Animals were randomized to treatments and pens using a program in SAS (SAS Release 9.4 or higher) which uses the ranuni function to generate random numbers. Animals were randomly allocated to treatment groups within block and batched based on pre-study pruritus scores and pen location. Blocks had three to four animals. There was one T01 animal in each block. Two T01 animals were randomly allocated to each of three batches completely at random. Within a batch, animals were randomly assigned to stalls. The following information was included in the randomization.

| | | | | | | |
|---|---|---|---|---|---|---|
| Animal* | Batch | Pen | Pre-Study | Block | Treatment | Treatment |
| | * | * | Pruritis Score | | | Description |

*Columns available to all study personnel.

### Inclusion/Exclusion Criteria

Animals were in overall good health and deemed suitable for the study. Horses had not received a non-steroidal anti-inflammatory drug within 7 days, short acting corticosteroid within 14 days, intermediate/long-acting or repository corticosteroids within 30 days. Animals with concurrent disease or that appear unthrifty, affecting the conduct of the study or the welfare of the animal were excluded from enrollment or from the study.

### Animal Weights

Animals were weighed within 14 days of dosing.

### Fasting

Animals were not fasted.

### Dosing Method

| Treatment | Route | Dosing Method |
|---|---|---|
| Placebo/ | Oral | Oclacitinib maleate tablets were fed orally |
| Oclacitinib maleate | | combined with carrier. Placebo group only |
| Tablets | | received carrier. |
| IL-31 Challenge | IV | IL-31 challenge was administered intrave- |
| Material | | nously to each animal via jugular vein. |

### Pre-Observation Acclimation

On the scheduled observation days, horses were acclimated to observation stalls for at least 1 hour prior to observation.

### Evaluation for Dermatological Lesions

A veterinarian evaluated horses for active dermatologic lesions on the day of IL-31 challenge. Active lesions can include, but are not limited to, open wounds, wheals, urticaria, papules and ulcerations. Any lesions were documented. If the lesion is severe, the horse may be excluded from study.

### Characterization of Pruritic Behavior

In order to characterize pruritic behavior, horses were observed for any behavior that can be identified as pruritus. Signs of pruritus in horses include:
Biting or scratching at self
Rubbing against objects
Feet stomping
Tail flicking
Head or body shaking
Rolling
Twitching of skin

A “yes” response was indicated by marking a “1” in the space provided for the specific behavior during the one minute interval. The cumulative number of yes responses per behavior were added to determine a cumulative pruritus score for each horse.

### Post-Challenge Observation Period

Following IL-31 challenge, horses were observed for 120 minutes to determine a post challenge pruritus score.

### Pharmacokinetics (PK)

Samples for PK were collected according to the protocol below. Tubes were gently inverted sufficiently to aid in the mixing of blood and anticoagulant. Samples were kept chilled on wet ice during collection and during processing. Plasma was stored frozen 10° C.

Blood for Pharmacokinetic Analysis

| | |
|---|---|
| Plasma: | Blood sample volume: approximately 2.0 mL |
| | Collection method: jugular venipuncture |
| | Tubes: K3 EDTA anticoagulant |
| | Processing: Place tube on ice after collection. Centri- |
| | fuge to effect separation of plasma within 1 hour; |
| | transfer single aliquot to 1.4 mL matrix tube for |
| | storage at ≤−10° C. until analysis. |
| Collection | Prior to IL-31 administration and immediately follow- |
| times: | ing challenge observation period. |
| Time Post Test | T01*-1.5 Hr, 4 Hr, 21.5 Hr and 24 Hr |
| Article Dosing | T02-1.5 Hr and 4 Hr |
| | T03-21.5 Hr and 24 Hr |
| | T04-4.5 Hr and 7 Hr |

*Not all T01 horses had blood collections at all time points. See batches in Section 4.8.
Oclacitinib was quantitated in plasma samples using an LC-MS/MS method.

### Results

Pruritic scoring shown as raw scores (Table 10):

TABLE 10
Raw Pruritis Scores for Horses Treated with Oclacitinib
Maleate Prior to IL-31 Challenge

| | | | Treat- | | Post | |
|---|---|---|---|---|---|---|
| Animal | | Batch | ment | Historical | Treatment | % |
| ID | Date | # | Group | Score | Score | Change |
| 595 | Sep. 11, 2018 | 1 | T02 | 67.5 | 46 | −31.85% |
| 51 | Sep. 11, 2018 | 1 | T01 | 65 | 112 | 72.31% |
| 571 | Sep. 11, 2018 | 1 | T02 | 67.5 | 21 | −68.89% |
| 630 | Sep. 11, 2018 | 1 | T02 | 88.5 | 94 | 6.21% |
| 599 | Sep. 11, 2018 | 1 | T01 | 89 | 106 | 19.10% |
| 591 | Sep. 11, 2018 | 1 | T02 | 96.5 | 91 | −5.70% |
| 596 | Sep. 11, 2018 | 1 | T02 | 95 | 86 | −9.47% |
| 593 | Sep. 12, 2018 | 2 | T03 | 51 | 49 | −3.92% |
| 597 | Sep. 12, 2018 | 2 | T03 | 54.5 | 90 | 65.14% |
| 573 | Sep. 12, 2018 | 2 | T01 | 64.5 | 96 | 48.84% |
| 585 | Sep. 12, 2018 | 2 | T03 | 73 | 86 | 17.81% |
| 55 | Sep. 12, 2018 | 2 | T03 | 70.5 | 50 | −29.08% |
| 638 | Sep. 12, 2018 | 2 | T01 | 71 | 67 | −5.63% |
| 589 | Sep. 12, 2018 | 2 | T03 | 76.5 | 72 | −5.88% |
| 579 | Sep. 13, 2018 | 3 | T04 | 109 | 49 | −55.05% |
| 594 | Sep. 13, 2018 | 3 | T04 | 112 | 87 | −22.32% |
| 592 | Sep. 13, 2018 | 3 | T01 | 111.5 | 108 | −3.14% |
| 598 | Sep. 13, 2018 | 3 | T04 | 100.5 | 46 | −54.23% |
| 583 | Sep. 13, 2018 | 3 | T04 | 98 | 53 | −45.92% |
| 619 | Sep. 13, 2018 | 3 | T04 | 105.5 | 99 | −6.16% |
| 639 | Sep. 13, 2018 | 3 | T01 | 107 | 93 | −13.08% |

### Discussion of Results of Example 4

Horses challenged with IL-31 following 0.25 mg/kg oclacitinib maleate treatment showed a reduction in pruritic activity when compared to the placebo horses. Horses challenged 1.5 hours after oclacitinib maleate treatment (T02) had a reduction in average pruritic score by 30% when compared to the placebo group (T01). Horses challenged 21.5 hours after oclacitinib maleate treatment (T03) had a reduction in average pruritic score by 29% when compared to the placebo group (T01). Horses challenged 4.5 hours after oclacitinib maleate treatment (T04) had a reduction in average pruritic score by 31% when compared to the placebo group (T01).

What is claimed is:

1. An IL-31 horse pruritus model comprising:
administering equine IL-31 to horses to produce a pruritic response;
quantitatively measuring pruritic responses in the horses which were administered equine IL-31;
administering a candidate horse IL-31 inhibitor; and
assessing the effectiveness of the candidate horse IL-31 inhibitor in reducing pruritic behavior in the treated horses by challenging the horses with equine IL-31 following the administration of the candidate horse IL-31 inhibitor.

2. The IL-31 horse pruritus model of claim 1, wherein the equine IL-31 is recombinant equine IL-31.

3. The IL-31 horse pruritus model of claim 2, wherein the recombinant equine IL-31 is encoded by a nucleic acid comprising the nucleotide sequence of SEQ ID NO:1.

4. The IL-31 horse pruritus model of claim 1, wherein the equine IL-31 is administered parenterally or intradermally.

5. The IL-31 horse pruritus model of claim 1, wherein the equine IL-31 is administered at a dose of 0.05 to 1 μg/kg.

6. The IL-31 horse pruritus model of claim 5, wherein the equine IL-31 is administered at a dose of 0.1 to 0.25 μg/kg.

7. The IL-31 horse pruritus model of claim 1, wherein the pruritic response is a transient response.

8. The IL-31 horse pruritus model of claim 7, wherein the transient pruritic response lasts less than 24 hours.

9. The IL-31 horse pruritus model of claim 1, wherein the pruritic responses in the horses are selected from the group consisting of biting or scratching at self, rubbing against objects, feet stomping, tail flicking, head or body shaking, rolling, twitching of skin, and combinations thereof.

10. The IL-31 horse pruritus model of claim 1, wherein pruritic behavior measurements are performed using real-time surveillance or video recording, using a categorical scoring system, or by timing pruritic events throughout an observation window.

11. The IL-31 horse pruritus model of claim 10, wherein at consecutive time intervals, “yes/no” decisions are made as to whether pruritic behavior was displayed by each horse.

12. The IL-31 horse pruritus model of claim 11, wherein the consecutive time intervals are 1 minute intervals.

13. The IL-31 horse pruritus model of claim 11, wherein at the end of a designated observation period, the numbers of yes determinations are added together to come up with a cumulative pruritic score (PS)

14. The IL-31 horse pruritus model of claim 13, wherein a first PS measurement is a baseline score measured immediately prior to the equine IL-31 challenge.

15. The IL-31 horse pruritus model of claim 14, wherein an additional PS measurement is determined following the equine IL-31 challenge.

16. The IL-31 horse pruritus model of claim 1, where the candidate horse IL-31 inhibitor comprises a small molecule compound, an anti-IL-31 antibody, or an IL-31 vaccine mimotope.

17. The IL-31 horse pruritus model of claim 16, wherein the candidate horse IL-31 inhibitor comprises a small molecule compound.

18. The IL-31 horse pruritus model of claim 17, wherein the small molecule compound is an inhibitor of the janus kinase pathway, the mitogen activated protein kinase pathway, or the Akt/protein kinase B pathway.

19. The IL-31 horse pruritus model of claim 17, wherein the small molecule compound is combined with a pharmaceutically acceptable carrier.

20. The IL-31 horse pruritus model of claim 19, where the carrier is a feed carrier.

21. The IL-31 horse pruritus model of claim 16, wherein the candidate horse IL-31 inhibitor is an IL-31 vaccine mimotope.

22. The IL-31 horse pruritus model of claim 21, wherein the IL-31 vaccine mimotope is an equine IL-31 mimotope, a feline IL-31 mimotope, or a canine IL-31 mimotope.

23. The IL-31 horse pruritus model of claim 21, where the IL-31 vaccine mimotope is a constrained mimotope or a linear mimotope.

24. The IL-31 horse pruritus model of claim 23, wherein the constrained mimotope is a chemically-linked cyclic peptide.

25. The IL-31 horse pruritus model of claim 21, wherein the IL-31 vaccine mimotope is combined with a carrier polypeptide.

26. The IL-31 horse pruritus model of claim 25, wherein the IL-31 vaccine mimotope is chemically conjugated to the carrier polypeptide.

27. The IL-31 horse pruritus model of claim 25, wherein the carrier polypeptide and the IL-31 mimotope are part of a recombinant fusion protein.

28. The IL-31 horse pruritus model of claim 25, wherein the carrier polypeptide includes a bacterial toxoid or a derivative thereof, keyhole limpet hemocyanin (KLH), a virus-like particle, or combinations thereof.

29. The IL-31 horse pruritus model of claim 28, wherein the bacterial toxoid or derivative is selected from tetanus toxoid, a diphtheria toxoid, a tetanus toxoid, the outer membrane protein complex from group B N. meningitidis, Pseudomonas exotoxin, or the nontoxic mutant of diphtheria toxin (CRM197).

30. The IL-31 horse pruritus model of claim 28, wherein the virus-like particle is selected from HBsAg, HBcAg, E. coli bacteriophage Qbeta, Norwalk virus, canine distemper virus (CDV), influenza HA, or cucumber mosaic virus (CuMV).

31. The IL-31 horse pruritus model of claim 21, wherein the IL-31 vaccine mimotope is combined with an adjuvant selected from the group consisting of an oil-in-water adjuvant, a polymer and water adjuvant, a water-in-oil adjuvant, an aluminum hydroxide adjuvant, a vitamin E adjuvant and combinations thereof.

32. The IL-31 horse pruritus model of claim 21, wherein the IL-31 vaccine mimotope is combined with an adjuvant formulation comprising a saponin, a sterol, a quaternary ammonium compound, and a polymer.

33. The IL-31 horse pruritus model of claim 32, wherein the saponin is Quil A or a purified fraction thereof, the sterol is cholesterol, the quaternary ammonium compound is dimethyl dioctadecyl ammonium bromide (DDA), and the polymer is polyacrylic acid.

34. The IL-31 horse pruritus model of claim 21, wherein the IL-31 vaccine mimotope is combined with an adjuvant comprising the combination of one or more isolated immunostimulatory oligonucleotides, a sterol, and a saponin.

35. The IL-31 horse pruritus model of claim 34, wherein the one or more isolated immunostimulatory oligonucleotides comprises CpG, the sterol is cholesterol, and the saponin is Quil A or a purified fraction thereof.

36. The IL-31 horse pruritus model of claim 21, wherein the IL-31 vaccine mimotope is combined with an adjuvant comprising an oil-in-water adjuvant containing one or more immunostimulatory oligonucleotides.

37. The IL-31 horse pruritus model of claim method of claim 16, wherein the candidate equine IL-31 inhibitor comprises an isolated IL-31 antibody or antigen-binding portion thereof.

38. The IL-31 horse pruritus model of claim 37, where the candidate horse IL-31 inhibitor is an equinized or fully equine monoclonal antibody.

39. The IL-31 horse pruritus model of claim 1, wherein the candidate horse IL-31 inhibitor is administered parenterally.

40. The IL-31 horse pruritus model of claim 1, wherein the candidate horse IL-31 inhibitor is administered orally.
